(19) United States
(12) Patent Application Publication
(10) Pub. No.: US 2020/0023363 A1
(43) Pub. Date: Jan. 23, 2020

(54) FLUIDIC SYSTEMS INCLUDING VESSELS AND RELATED METHODS

(71) Applicant: ArcherDX, Inc., Boulder, CO (US)

(72) Inventors: Joshua Stahl, Boulder, CO (US); Jason Myers, Boulder, CO (US)

(73) Assignee: ArcherDX, Inc., Boulder, CO (US)

(21) Appl. No.: 16/336,353

(22) PCT Filed: Sep. 22, 2017
(86) PCT No.: PCT/US2017/053108

§ 371 Date: Mar. 25, 2019

Related U.S. Application Data
(63) Continuation of application No. PCT/US2017/051924, filed on Sep. 15, 2017.
Application No. PCT/US2017/051924 is a continuation of application No. PCT/US2017/051927, filed on Sep. 15, 2017.
(60) Provisional application No. 62/398,841, filed on Sep. 23, 2016.
(60) Provisional application No. 62/399,152, filed on Sep. 23, 2016.
(60) Provisional application No. 62/399,157, filed on Sep. 23, 2016.
(60) Provisional application No. 62/399,184, filed on Sep. 23, 2016.
(60) Provisional application No. 62/399,195, filed on Sep. 23, 2016.
(60) Provisional application No. 62/399,205, filed on Sep. 23, 2016.
(60) Provisional application No. 62/399,211, filed on Sep. 23, 2016.
(60) Provisional application No. 62/399,219, filed on Sep. 23, 2016.

(51) Int. Cl. B01L 3/00; B01L 7/00; C40B 30/06; G01N 1/34; G01N 21/64; G01N 30/04; G01N 33/543; G01N 35/00; G01N 35/02; G16B 25/20; G16B 30/00; G16B 35/10

(52) U.S. Cl. CPC B01L 3/502761; G01N 2030/8827; B01L 3/50825; B01L 7/52; C40B 30/06; G01N 1/34; G01N 21/6428; G01N 30/04; G01N 33/54326; G01N 35/00732; G01N 35/0098; G01N 35/026; G16B 25/20; G16B 30/00; G16B 35/10; B01L 2200/027; B01L 2200/0668; B01L 2200/16; B01L 2300/021; B01L 2300/041; B01L 2300/0816; B01L 2300/0887; B01L 2300/12; B01L 2300/1822; B01L 2400/043; B01L 2400/0644; B01L 3/502738

(57) ABSTRACT

Fluidic systems including cartridges comprising vessels, microfluidic channels and related methods for performing chemical and/or biological analyses are generally provided. The systems described herein include, according to certain embodiments, a cartridge comprising a vessel adapted and arranged to contain a fluid and/or reagent for performing a chemical and/or biological analysis. The vessel may be designed to have a particular shape or configuration, such as a tapered cross-sectional shape, e.g., to facilitate manipulation of a fluid and/or reagent within the vessel (e.g., a lyosphere).

### RELATED APPLICATIONS

This application claims priority under 35 U.S.C. § 119(e) to U.S. Provisional Patent Application Nos. 62/398,841, 62/399,152, 62/399,157, 62/399,184, 62/399,195, 62/399,205, 62/399,211, and 62/399,219, each of which was filed on Sep. 23, 2016, and claims priority under 35 U.S.C. §§ 120 and 365(c) to PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and to PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which applications are hereby incorporated by reference.

### TECHNICAL FIELD

The present invention generally relates to systems and related methods for automated processing of molecules (e.g., nucleic acids). The present invention also generally relates to fluidic systems and related methods, the systems comprising cartridges including vessels and/or microfluidic channels.

### BACKGROUND

Numerous approaches for processing nucleic acids have been developed. Such methods often included multiple enzymatic, purification, and preparative steps that make them laborious and prone to error, including errors associated with contamination, systematic user errors, and process biases. As a result, it is often difficult to execute such processes reliably and reproducibly, particularly when the processes are being conducted commercially, e.g., in a multiplex or high-throughput context.

### SUMMARY

The present invention generally relates to systems and related methods for processing nucleic acids. In some embodiments, the system comprises cartridges including cassettes and/or microfluidic channels that facilitate automated processing of nucleic acids, including automated nucleic acid library preparations. In some embodiments, systems and related methods are provided for automated processing of nucleic acids to produces material for next generation sequencing and/or other downstream analytical techniques. The present invention generally relates to fluidic systems and related methods, the systems comprising cartridges including vessels and/or microfluidic channels.

In one set of embodiments, a series of cartridges are provided. In one embodiment, a cartridge comprises a vessel comprising an inlet and a tapered cross-sectional shape, wherein the vessel has an internal working volume of at least 5 μl and less than or equal to 70 μl; a microfluidic channel in fluid communication with the vessel for delivering a fluid to the vessel; and an orifice positioned between the microfluidic channel and the vessel proximate the inlet to the vessel, wherein the orifice has a cross-sectional dimension of at least 10 microns and less than or equal to 500 microns.

In another embodiment, a cartridge comprises a vessel comprising an inlet; an orifice positioned proximate the inlet to the vessel, wherein the orifice has a cross-sectional dimension of at least 10 microns and less than or equal to 500 microns; a vent channel in fluid communication with the vessel and configured to receive a gaseous fluid from the vessel; and a gas-permeable membrane configured to allow air to pass through the membrane while substantially preventing a liquid or vapor from passing across the membrane, wherein the vent channel is positioned between the gas-permeable membrane and the vessel.

In another embodiment, a cartridge comprises a vessel having an internal working volume of at least 5 μl and less than or equal to 70 μl, wherein the vessel has a longest dimension positioned along a first plane; a microfluidic channel in fluid communication with the vessel and configured to deliver a fluid to the vessel, wherein the microfluidic channel has a longest dimension along a second plane, and wherein the first plane is substantially perpendicular to the second plane.

In another set of embodiments, methods are provided. In one embodiment, a method for performing a reaction is provided. The method comprises flowing a first fluid comprising a first reagent in a microfluidic channel; introducing at least a portion of the first fluid to a vessel having an internal working volume and containing a second reagent to fill a portion, but not all, of the internal working volume of the vessel with the first fluid; and reacting the first reagent with the second reagent, wherein during the reaction a ratio by volume of liquid to a gaseous fluid in the reaction vessel is at least 1 to 5 and less than or equal to 5 to 1.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic drawing of a nucleic acid library preparation workflow;

FIG. 2A is a drawing of a system for automated nucleic acid library preparation using a microfluidic cartridge;

FIG. 2B is a drawing showing internal components of a system for automated nucleic acid library preparation using a microfluidic cartridge;

FIG. 3 is a perspective view of a microfluidic cartridge bay assembly;

FIG. 4A is a top view of a microfluidic cartridge carrier assembly;

FIG. 4B is a perspective view of a microfluidic cartridge;

FIG. 5 is an exploded view of a microfluidic cartridge;

FIG. 6A is a side view of a system including a vessel connected to a microfluidic channel;

FIG. 6B is a perspective view of an inlet of a vessel separated from an orifice;

FIG. 7A is a side view of a system including a series of vessels connected to a microfluidic channel;

FIG. 7B is a side view of a system including a series of vessels connected to a series of microfluidic channels;

FIG. 7C is a side view of another system including a series of vessels connected to a series of microfluidic channels;

FIG. 7D is a side view of a system including a series of vessels;

FIG. 8A is a side view of a vessel containing a lyosphere and connected to a microfluidic channel;

FIG. 8B is a side view of a vessel containing a lyosphere and a fluid reagent;

FIG. 9 is a top view of a channel system including a series of fluidic channels connected to vessels and other components;

FIG. 10 is a perspective view showing layers of a microfluidic cartridge; and

FIG. 11 is a side view of a vessel connected to a channel system.

### DETAILED DESCRIPTION

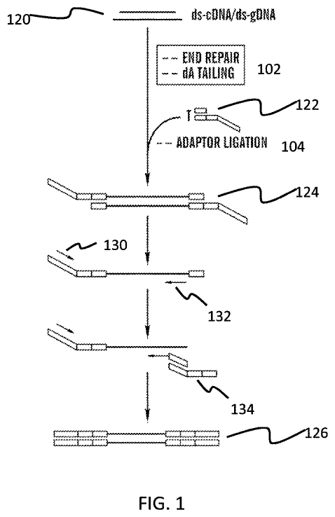

Systems including cartridges with modular components (cassettes) and/or microfluidic channels for processing nucleic acids are generally provided. In some embodiments, systems and related methods are provided for automated processing of nucleic acids to produce material for next generation sequencing and/or other downstream analytical techniques. In some embodiments, systems described herein include a cartridge comprising, a frame, one or more cassettes which may be inserted into the frame, and a channel system for transporting fluids. In certain embodiments, the one or more cassettes comprise one or more reservoirs or vessels configured to contain and/or receive a fluid (e.g., a stored reagent, a sample). In some cases, the stored reagent may include one or more lyospheres. The systems and methods described herein may be useful for performing chemical and/or biological reactions including reactions for nucleic acid processing, including polymerase chain reactions (PCR). In some embodiments, systems and methods provided herein may be used for processing nucleic acids as depicted in FIG. 1. For example, in some embodiments, the nucleic acid preparation methods depicted in FIG. 1, which are described in greater detail herein, may be conducted in a multiplex fashion with multiple different (e.g., up to 8 different) samples being processed in parallel in an automated fashion. Such systems and methods may be implemented within a laboratory, clinical (e.g., hospital), or research setting.

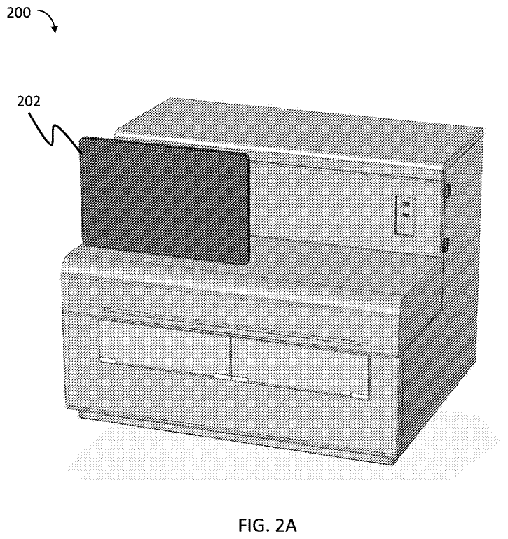

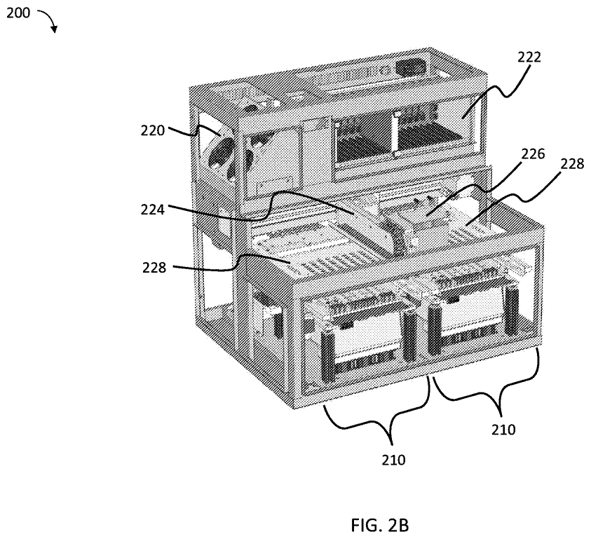

In some embodiments, systems provided herein may be used for next generation sequencing (NGS) sample preparation (e.g., library sample preparation). In some embodiments, systems provided herein may be used for sample quality control. FIGS. 2A and 2B depict an example system 200 which serves as a laboratory bench top instrument which utilizes a number of disposable cassettes, primer cassettes, and bulk fluid cassettes. In some embodiments, this system is suitable for use on a standard laboratory workbench. In some embodiments, a system may have a touch screen interface (e.g., as depicted in the exemplary system of FIG. 2A comprising a touch screen interface 202). In some embodiments, the interface displays the status of each of the one or more cartridge bays with “estimated time to complete”, “current process step”, or other indicators. In some embodiments, a log file or report may be created for each of the one or more cartridges. In some embodiments, the log file or report may be saved on the instrument. In some embodiments, a text file or output may be sent from the instrument, e.g., for a date range of cartridges processed or for a cartridge with a particular serial number.

In some embodiments, systems provided herein may comprise one or more cartridge bays (e.g., two, as depicted in the exemplary system of FIG. 2B comprising two cartridge bays 210), capable of receiving one or more nucleic acid preparation cartridges. In some embodiments, a space above the cartridge bay(s) is reserved for an XY positioner 224 to move an optics module 226 (and/or a barcode scanner, e.g., a 2-D barcode scanner) above lids 228 (e.g., heated lids) of each cartridge bay. In some embodiments, the system comprises an electronics module 222 that drives optics module 226 and XY positioner 224. In some embodiments, XY positioner 224 will position optics module 226 such that it can excite materials (e.g., fluorophores) in the vessel and collect the emitted fluorescent light. In some embodiments, this will occur through holes placed in the lid (e.g., heated lid) over each vessel. In some embodiments, a barcode scanner will confirm that appropriate cartridge and primer cassettes have been inserted in the system. In some embodiments, optics module 226 will collect light signals from each cartridge in each cartridge bay, as needed, during processing of a sample, e.g., during amplification of a nucleic acid to detect the level of the amplified nucleic acid. In some embodiments, the systems described herein comprise elements that assist in temperature regulation of components within the system, such as one or more fans or fan assemblies (e.g., the fan assembly 220 depicted in FIG. 2B).

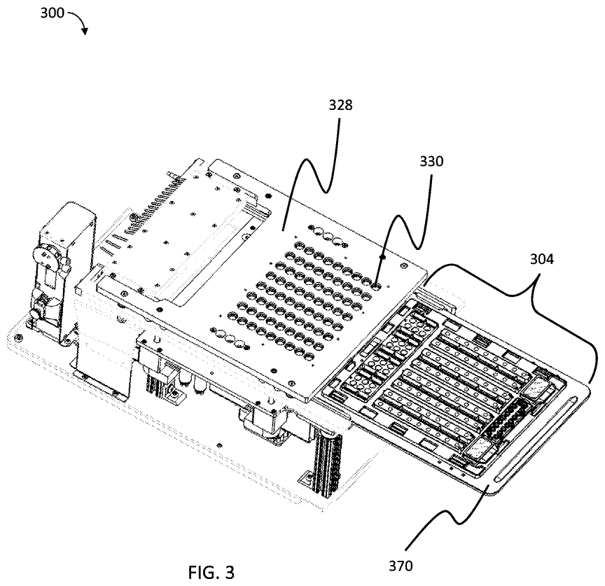

In some embodiments, the one or more cartridge bays can process nucleic acid preparation cartridges, in any combination. In some embodiments, each cartridge bay is loaded, e.g., by the operator or by a robotic assembly. FIG. 3 depicts an exemplary drawing of a microfluidics cartridge bay assembly 300. In some embodiments, a cartridge is loaded into a bay when the bay is in the open position by placing the cartridge into a carrier plate 370 to form a carrier plate assembly 304. The carrier plate is itself, in some embodiments, a stand-alone component which may be removed from the cartridge bay. This cartridge bay holds the cartridge in a known position relative to the instrument. In some embodiments, a lid 328 (e.g., a heated lid) comprises one or more holes 330 to facilitate the processing and/or monitoring of reactions occurring in one or more vessels. In some embodiments, prior to loading a new cartridge onto the instrument, a primer cassette may be installed onto the cartridge. In some embodiments, the primer cassette would be packaged separately from the cartridge. In some embodiments, a primer cassette may be placed into a cartridge. In some embodiments, both primer cassettes and cartridges would be identified such that placing them onto the instrument allows the instrument to read them (e.g., using a barcode scanner) and initiate a protocol associated with the cassettes.

In some embodiments, prior to installing a carrier into the instrument, bulk reagents may be loaded into the carrier. In some embodiments, a user or robotic assembly may be informed as to which reagents to load and where to load them by the instrument or an interface on a remote sample loading station. In some embodiments, after loading a cartridge with a primer cassette into an instrument, a user would have the option of choosing certain reaction conditions (e.g., a number of PCR cycles) and/or the quantity of samples to be run on the cartridge. In some embodiments, each cartridge may have a capacity of 1, 2, 3, 4, 5, 6, 7, 8, 9, 10 or more samples.

In some embodiments, systems provided herein may be configured to process RNA. However, in some embodiments, the system may be configured to process DNA. In some embodiments, different nucleic acids may be processed in series or in parallel within the system. In some embodiments, cartridges may be used to perform gene fusion assays in an automated fashion, for example, to detect genetic alterations in ALK, RET, or ROS1. Such assays are disclosed herein as well as in U.S. Patent Application Publication No. 2013/0303461, which was published on Nov. 14, 2013, U.S. Patent Application Publication No. and 2015/02011050, which was published on Jul. 20, 2013, the contents of each of which are incorporated herein by reference in their entirety. In some embodiments, systems provided herein can process in an automated fashion an Xgen protocol from Integrated DNA Technologies or other similar nucleic acid processing protocol. In some embodiments, cartridge and cassettes will have all of the reagents needed for carrying out a particular protocol. In some embodiments, once a carrier is loaded into a cartridge bay an access door to that bay is closed, and optionally a lid (e.g., a heated lid) may be lowered automatically. In some embodiments, lowering of the lid (e.g., the heated lid) forces (or places) the cartridge down onto an array of heater jackets which conform to each of a set of one or more temperature controlled vessels in the cartridge. In some embodiments, this places the cartridge in a known position vertically in the drawer assembly. In some embodiments, lowering of the lid forces the cartridge down into a position in which rotary valves present in the cartridge are capable of engaging with corresponding drivers that control the rotational position of the valves in the cartridge. In some embodiments, automation components are provided to ensure that the rotary valves properly engage with their drivers.

In some embodiments of methods provided herein, a nucleic acid sample present in a cartridge (e.g., within a vessel of a cassette) will be mixed with a lyosphere. In some embodiments, the lyosphere will contain a fluorophore which will attach to the sample. In some embodiments, there will also be a “reference material” in the lyosphere which will contain a known amount of a molecule (e.g., of synthetic DNA). In some embodiments, another hybridization or hydrolysis probe will bind specifically to the “reference material”. In some embodiments, the aforementioned probe will have separate spectral properties of absorption and emission relative to the sample's fluorophore. In some embodiments, attached to the “reference material” will be another fluorophore which will emit light at a different wavelength than the sample's fluorophore. In some embodiments, a lyosphere will contain a fluorescent hybridization or hydrolysis probe that will bind to a sample or specific targets in the sample. In some embodiments, fluorophores used may be attached to the sample or the “reference material” via an intercalating dye (e.g., SYBR Green) or a reporter/quencher chemistry (e.g., TaqMan, etc.). In some embodiments, during quantitative PCR (qPCR) cycling the fluorescence of the two fluorophores will be monitored and then used to determine the amount of nucleic acid (e.g., DNA, cDNA) in the sample by the Comparative CT method. In some embodiments, the comparative CT method is used to describe relative concentration to a housekeeping gene. In some embodiments, the reference is a synthetic molecule added at a known concentration, which act as an internal standard for absolute quantification. In some embodiments, two fluorophores will be monitored and then used to determine the amount of nucleic acid (e.g., DNA, cDNA) in the sample based on the signal from the reference material.

Advantageously, certain systems described herein may include modular components (e.g., cassettes) that can allow tailoring of specific reactions and/or steps to be performed. In some embodiments, certain cassettes for performing a particular type of reaction are included in the cartridge. For example, cassettes including vessels containing lyospheres with different reagents for performing multiple steps of a PCR reaction may be present in the cartridge. The frame or cartridge may further include empty regions for a user to insert one or more cassettes containing specific fluids and/or reagents for a specific reaction (or set of reactions) to be performed in the cartridge. For example, a user may insert one or more cassettes containing particular buffers, reagents, alcohols, and/or primers into the frame or cartridge. Alternatively, a user may insert a different set of cassettes including a different set of fluids and/or reagents into the empty regions of the frame or cassette for performing a different reaction and/or experiment. After the cassettes are inserted into the frame or cartridge, they may form a fluidic connection with a channel system for transporting fluids to conduct the reactions/analyses.

In some embodiments, multiple analyses may be performed simultaneously or sequentially by inserting different cassettes into the cartridge. For instance, the systems and methods described herein may advantageously provide the ability to analyze two or more samples without the need to open the system or change the cartridge. For example, in some cases, one or more reactions with one or more samples may be conducted in parallel (e.g., conducting two or more PCR reactions in parallel). Such modularity and flexibility may allow for the analysis of multiple samples, each of which may require one or several reaction steps within a single fluidic system. Accordingly, multiple complex reactions and analyses may be performed using the systems and methods described herein.

Unlike certain existing fluidic systems and methods, the systems and methods described herein may be reusable (e.g., a reusable carrier plate) or disposable (e.g., consumable components including cassettes and various fluidic components). In some cases, the systems described herein may occupy a relatively small footprint as compared to certain existing fluidic systems for performing similar reactions and experiments.

In some embodiments, the cassettes and/or cartridge includes stored fluids and/or reagents needed to perform a particular reaction or analysis (or set of reactions or analyses) with one or more samples. Examples of cassettes include, but are not limited to, reagent cassettes, primer cassettes, buffer cassettes, waste cassettes, sample cassettes, and output cassettes. Other appropriate modules or cassettes may be used. Such cassettes may be configured in a manner that prevents or eliminates contamination or loss of the stored reagents prior to the use of those reagents. Other advantages are described in more detail below.

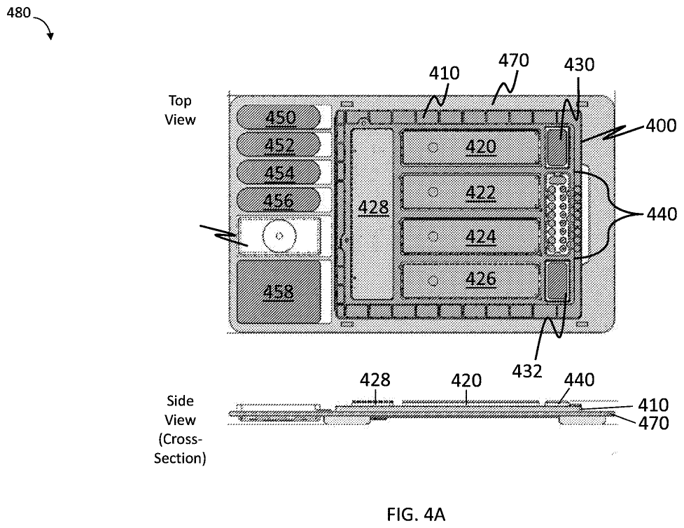

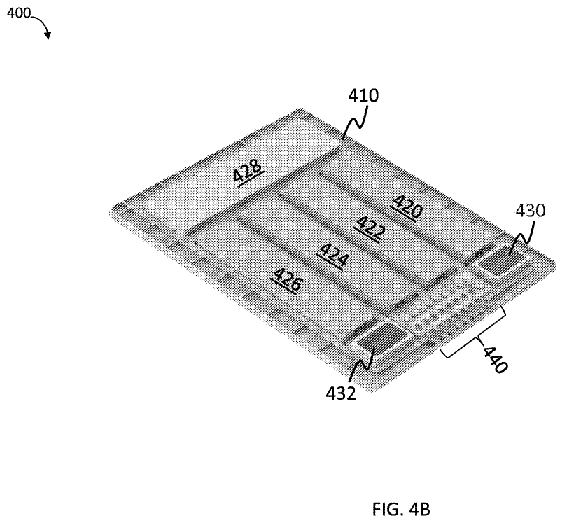

In one embodiment, as shown illustratively in FIGS. 4A and 4B, cartridge 400 comprises a frame 410 and cassettes 420, 422, 424, 426, 428, 430, 432, and 440. In some embodiments, each of these cassettes may be in fluidic communication with a channel system (e.g., positioned underneath the cassettes, not shown). In some embodiments, at least one of cassettes 428 (e.g., a reagent cassettes), 430 (e.g., a reagent cassette), and 432 (e.g., a reagent cassette) may be inserted into frame 410 by the user such that the cassettes are in fluidic communication with the channel system. For example, in some embodiments, one of cassettes 428, 430, and 432 is a reagent cassette containing a reaction buffer (e.g., Tris buffer). In certain embodiments, cassettes 428, 430 and/or 432 may comprise one or more reagents and/or reaction vessels for a reaction or a set of reactions. In some embodiments, module 440 comprises a plurality of sample wells and/or output wells (e.g., samples wells configured to receive one or more samples). In some cases, cassettes 420, 422, 424, and 426 may comprise one or more stored reagents or reactants (e.g., lyospheres). For instance, each of cassettes 420, 422, 424, and 426 may include different sets of stored reagents or reactants for performing separate reactions. For example, cassette 420 may include a first set of reagents for performing a first PCR reaction, and cassette 422 may include a second set of reagents for performing a second PCR reaction. The first and second reactions may be performed simultaneously (e.g., in parallel) or sequentially.

In some embodiments, as shown illustratively in FIG. 4A, a carrier plate assembly 480 comprises a carrier plate 470 and additional cassettes including modules 450, 452, 454, 456, 458, and 460. In an exemplary embodiment, cassettes 450, 452, 454, 456, 458, and 460 may each comprise one or more stored reagents and/or may be configured and arranged to receive one or more fluids (e.g., module 458 may be a waste module configured to collect reaction waste fluids). In some embodiments, one or more of cassettes 450, 452, 454, 456, 458, and 460 may be refillable.

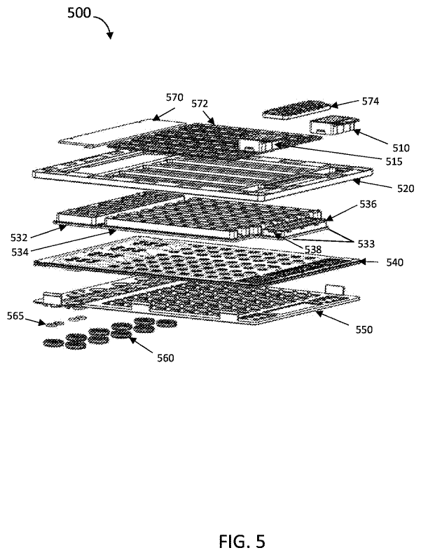

FIG. 5 is an exploded view of an exemplary cartridge 500, according to one set of embodiments. Cartridge 500 comprises a primer cassette 510 and a primer cassette 515 which may be inserted into one or more openings in a frame 520. Cartridge 500 further comprises a fluidics layer assembly 540 containing a channel system adjacent and non-integral to frame 520. In some embodiments, a set of cassettes 532 (e.g., comprising one or more primer cassettes, buffer cassettes, reagent cassettes, and/or waste cassettes, each optionally including one or more vessels), set of reaction cassettes 534, which comprises reaction vessels, an input/output cassette 533, which comprises sample input vessels 536 and output vessels 538, may be inserted into one or more openings in frame 520. In some embodiments, cartridge 500 comprises a valve plate 550. In some embodiments, valve plate 550 connects (e.g., snaps) into frame 520 and holds in place fluidics layer assembly 540 and cassettes 532, 533 and 534 in frame 520. In certain embodiments, cartridge 500 comprises valves 560, as described herein, and a plurality of seals 565. In some cases, frame 520 and/or one or more modules may be covered by covers 570, 572, and/or 574.

As described above, the systems described herein include, according to certain embodiments, a cartridge comprising a vessel adapted and arranged to contain a fluid and/or reagent for performing a chemical and/or biological analysis. The vessel may be designed to have a particular shape or configuration, such as a tapered cross-sectional shape, e.g., to facilitate manipulation of a fluid and/or reagent within the vessel (e.g., a lyosphere). A microfluidic channel connected to a channel system may be in fluid communication with the vessel. The channel system may be used to introduce and/or remove fluids and/or reagents into and from the vessel. In certain embodiments the cartridge further comprises a vent channel in fluid communication with the vessel, which allows a gaseous fluid to vent from the vessel. In certain embodiments the cartridge further comprises a gas-permeable membrane configured to allow air to pass through the membrane while substantially preventing a liquid or vapor from passing across the membrane. The vent channel may be positioned between the gas-permeable membrane and the vessel.

In some embodiments, the cartridge includes stored fluids and/or reagents that can be used to perform a particular reaction or analysis (or set of reactions or analyses) with one or more samples. In some cases, the stored fluids are contained within one or more reservoirs or vessels configured to contain and/or receive a fluid. In some embodiments, reservoirs or vessels may be present in one more modular components (e.g., cassettes, modules) that can be inserted and/or fixed to the cartridge and/or a frame of the cartridge. Examples of cassettes that may include one or more vessels include, but are not limited to, reagent cassettes, primer cassettes, buffer cassettes, waste cassettes, sample cassettes, and output cassettes. Cartridges may be configured in a manner that prevents or eliminates contamination or loss of the stored reagents prior to the use of those reagents. Other advantages are described in more detail below.

According to certain embodiments, the cartridge may be used to carry out one or more chemical reactions (e.g., reactions related to a polymerase chain reaction process). In one set of embodiments, for example, a method involving the cartridge comprise flowing a fluid comprising reagent (e.g., a sample fluid) from a source through the microfluidic channel. The fluid is then introduced to a vessel (e.g., tapered vessel), passing through an orifice first, according to certain embodiments. The fluid may fill a portion, but not all, of the internal working volume of the vessel. Inside the vessel is a second reagent (e.g., a dry reagent) which then mixes and reacts with the fluid that has been introduced, to produce the desired reaction for that application. In the process of reacting, vapor is generally formed. This vapor in conjunction with air already present in the vessel could cause undesired pressure build-up in the vessel. At the same time, venting the vapor could cause the loss of desired reaction product. According to certain embodiments, a selective gas permeable membrane allows for air to vent while substantially blocking the exit of vapor, thereby avoiding these two problems.

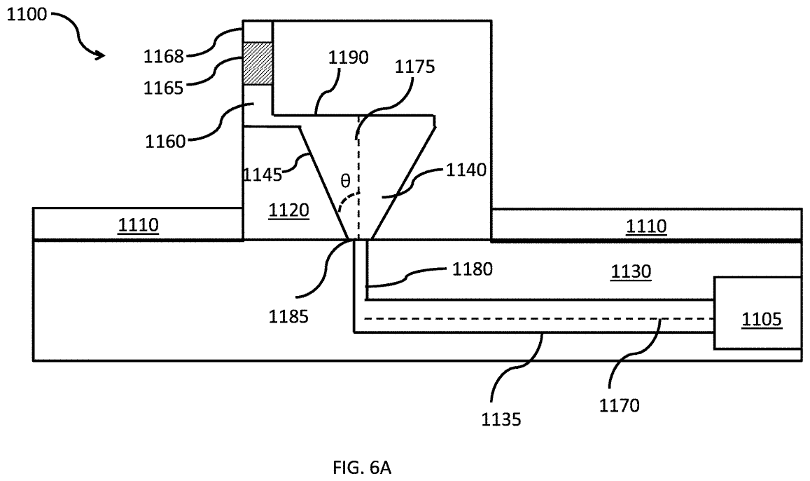

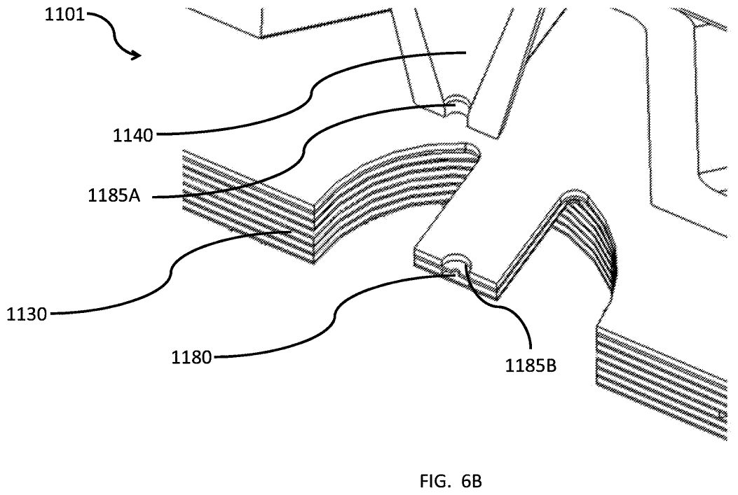

As described herein, a cartridge may include a frame surrounding one or more vessels and a channel system fluidically connected to the one or more vessels. In some embodiments, the one or more vessels may be part of one or more cassettes insertable into the frame of the cartridge and fluidically connected to a channel system. Various configurations of channels and valves in a channel system are depicted in FIGS. 6A-B, 7A-7D, and 8A-8B, and described in further detail below. One skilled in the art would appreciate that each configuration may share similar elements with other configurations. Accordingly, where a figure of this set lacks specific description for an element depicted in the figure, the element is as described in one or more of the other figures.

As shown illustratively in FIG. 6A, a portion of a cartridge 1100 comprises a frame 1110 and a channel system 1130 located below the frame 1110. Channel system 1130 comprises at least one fluidic (e.g., microfluidic) channel 1135. Channel systems and fluidic channels are described in more detail below. The fluidic channel 1135 is fluidly connected to vessel 1140. In some embodiments, the one or more vessels 1140 contain a fluid (e.g., a buffer) and/or a reagent. In the embodiment shown in FIG. 6A, vessel 1140 is positioned and/or fixed with a cassette 1120, however, the vessel 1140 could, alternatively, be integral with the frame and/or microfluidic channel system. Any fluids and/or reagents may be introduced into the vessel 1140 either after or before the cassettes 1120 are inserted into frame 1110 according to different embodiments. In some configurations, a reagent (e.g., a lyosphere) may be stored in vessel 1140 prior to use of the cartridge (e.g., prior to insertion of a sample into the cartridge). A fluid and/or reagent may be transported from the channel system 1130 (e.g., via a fluidic channel 1135 in the channel system 1130) to vessel 1140. For example, a fluid in channel 1135 may be transported to a first vessel 1140 in which a first reaction can take place. The resulting fluid, in some cases, may be transported back to the channel system 1130. In certain embodiments, the resulting fluid may be transported to a second vessel in which a second reaction can take place. In some embodiments, the cartridge 1100 may comprise a plurality of vessels 1140 configured and arranged such that a plurality of reactions make take place within the vessels.

In some embodiments, cassette 1120 (which may include one or more vessels) may be inserted into (e.g., positioned into) frame 1110, and may be adjacent to different components of the cartridge. For example, the cartridge may be configured such that the opening of the frame allows fluidic communication between the cassette and the channel system (e.g., a channel, port, or other fluidic component of the channel system).

In some embodiments, a cassette (which may include one or more vessels) may be inserted into the frame by a user. However, those skilled in the art would understand, based upon the teaching of this specification, that the cassette need not necessarily be inserted into the frame by the user. For example, in some embodiments, the cassette may already be present in the cartridge upon use of the cartridge (e.g., performing of a reaction within the cartridge) by the user. In certain embodiments, the cassette may be present in the cartridge upon and/or during fabrication of the cartridge (e.g., the cassette may be inserted into the frame/cartridge by the manufacturer). In some cases, the cassette may be physically connected to the cartridge. For example, in some such embodiments, the cassette may be connected to, and configured to maintain contact with, a surface of the channel system via an adhesive (e.g., an epoxy), a mechanical mechanism (e.g., a groove, a latch), friction, or by other means known in the art. Connection of the cassette to the cartridge may be conducted by the manufacturer and/or by the user, in some cases.

In the embodiment shown in FIG. 6A, a fluid and/or a reagent (e.g., a fluid sample) may be delivered to vessel 1140 from a source of fluid source 1105 (e.g., sample wells) via microfluidic channel 1135. The fluid travels through the microfluidic channel through an orifice 1180 which has a smaller (average) diameter as compared to the (average) diameters of the microfluidic channel 1135 and the (average) diameter of an inlet 1185 of the vessel 1140. For example, in some instances the orifice may have a diameter and/or length of less than 100 microns (e.g., less than or equal to 75 microns, 50 microns), while the diameter and/or length of channel 1135 may be larger than 100 microns. Other dimensions are possible and described herein. The orifice 1180 at the bottom of the vessel aids in keeping the fluid in the vessel 1140 during pressure and temperature changes in the vessel. In some embodiments, the orifice is integral to the inlet of the vessel. In some cases, the orifice may be an opening having the smallest cross-sectional dimension of the inlet of the vessel. Valving (not shown) may also aid in preventing backflow. In some embodiments, one or more valves may be positioned between channel 1135 and fluid source 1105, or on a side of the fluid source opposite channel 1135.

After the fluid enters vessel 1140 through inlet 1185, the fluid may react with a reagent present in the vessel 1140. While a reaction is taking place, fluid, air, and vapor occupy the volume of the reactor 1140, the volume of which is defined, at least in part, by the sidewall 1145 and a vessel cover 1190. A selective gas-permeable membrane 1165 allows for air to be vented from the cartridge 1100 while blocking all or most vapor present in the vessel (e.g., as a result of heating of the vessel). The presence of membrane 1165 advantageously allows for an appropriate pressure to be maintained or reached in the vessel while keeping to a minimum the amount of mass of reactants lost from the system. A vent channel 1160 receives gaseous fluid from the vessel 1140 and is positioned between the gas-permeable membrane 1165 and the vessel 1140. An outlet channel 1168 may be positioned downstream of the gas-permeable membrane 1165.

As shown in FIG. 6A, the vessel 1140 may have a tapered cross-sectional shape defined by sidewall 1145. The vessel's cross-sectional diameter expands in the direction away from the inlet 1185. The tapered (e.g., cylindrical) shape of the vessel results in the absence of ledges within the well, thereby facilitating better mixing and avoidance of residue.

As shown in FIG. 6A, the vessel 1140 has a longest dimension along a plane represented by a dashed line 1175. Likewise, the microfluidic channel 1135 has a longest dimension along a second plane represented by a dashed line 1170. In the embodiment shown in FIG. 6A, the plane represented by dashed line 1175 is substantially perpendicular to (and vertically oriented to) the plane represented by dashed line 1170.

FIG. 6B shows an exploded view of a portion of a system 1101 similar to the system shown in FIG. 6A, but in a configuration in which the vessel is separated from the orifice. As shown illustratively in this figure, orifice 1180 positioned in a fluidic channel layer 1130 has a reduced-width (average) diameter (e.g., an (average) diameter smaller than that of the inlet 1185 to the vessel 1140. The inlet 1185 has a top portion 1185A and a bottom portion 1185B that leads into the vessel 1140. It should be appreciated that while the orifice shown in this figure is formed in a layer separate from layer(s) forming the vessel, in other embodiments the orifice is integral to the vessel (e.g., integral to an inlet of the vessel). In some cases, the orifice may be an opening of the vessel, the opening having the smallest cross-sectional dimension of the inlet of the vessel (e.g., an inlet connected to a microfluidic channel of the channel system). In some cases, the orifice may form the narrowest portion, or the portion having the smallest cross-sectional dimension, of any fluid path between the vessel and the microfluidic channel of the channel system.

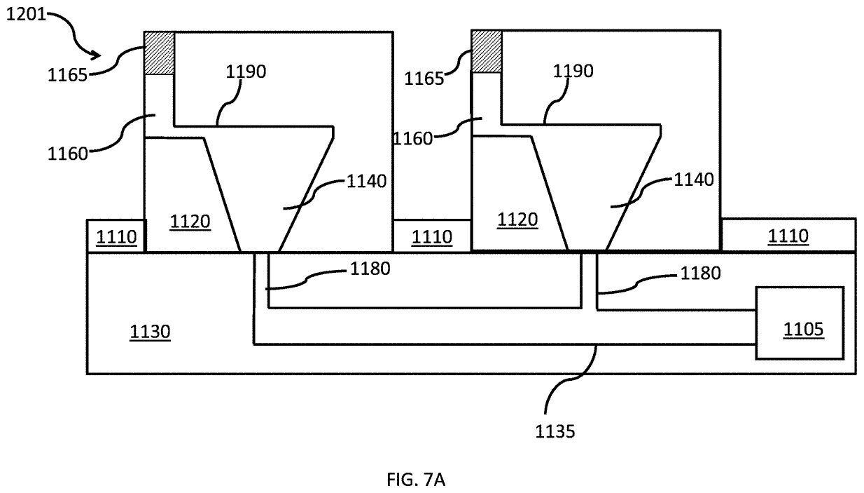

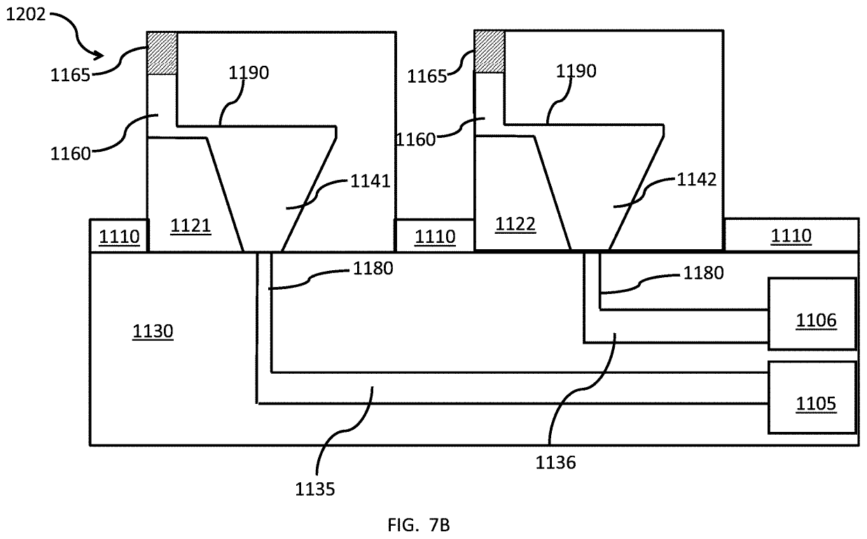

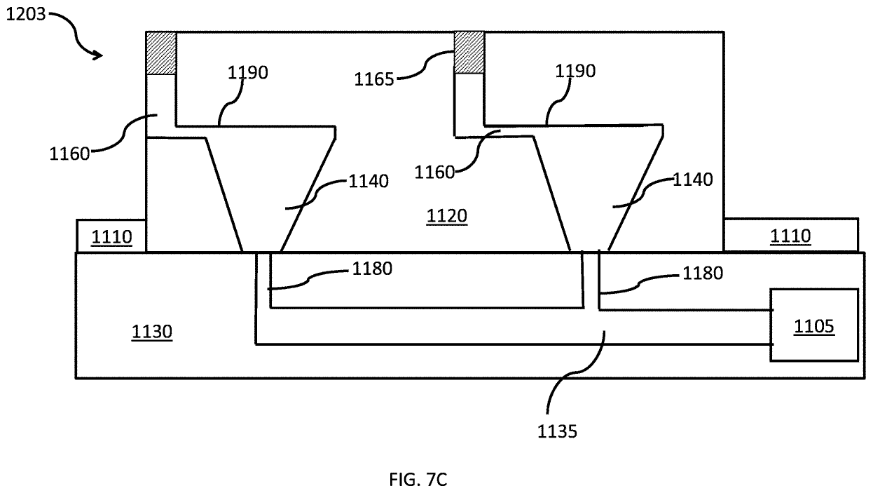

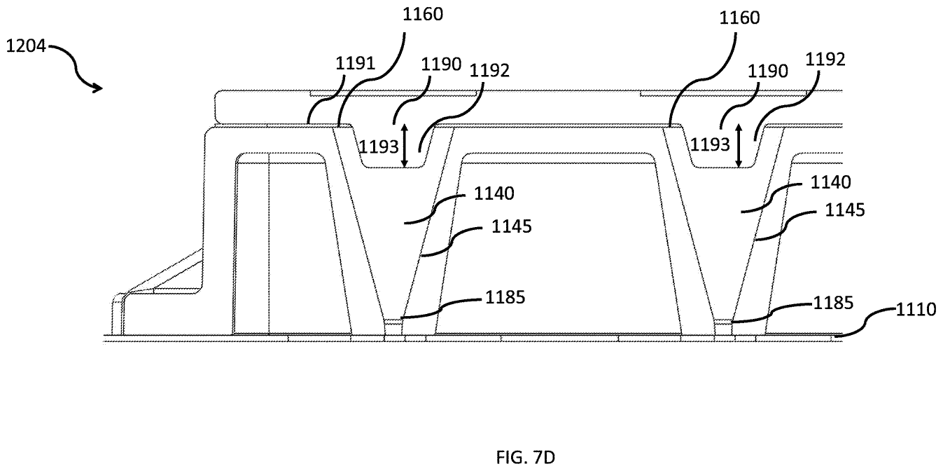

In certain embodiments, as shown illustratively in FIG. 7A, a cartridge 1201 comprises a frame 1110 having two or more openings each configured to receive a cassette 1120 including one or more vessels 1140. Alternatively, the vessels may generally have the same (or a different) configuration but be formed integral with the frame 1110. In the embodiment shown in FIG. 7A, the vessels are positioned in series, each fluidly connected to a source of fluid 1105 (e.g., sample wells) via microfluidic channel 1135. The same or different reagents may be present in the different vessels 1140. However, the general operation and structure of the vessel 1140 and the components surrounding each of the vessels 1140 (e.g., orifice 1180, gas-permeable membrane 1165) are the same as in FIG. 6A. Alternatively, as shown in FIG. 7B, according to one or more embodiments, the different vessels 1141 and 1142 of a cartridge 1202 may be fluidly connected to separate fluid sources (e.g., sample wells) 1105 and 1106, via microfluidic channels 1135 and 1136, respectively. Furthermore, the difference vessels 1141 and 1142 (included in cassettes 1121 and 1122, respectively) may contain different reagents depending on the desired reactions (e.g., a first reagent for conducting a first reaction in the first vessel 1141 and a second reagent for conducting a second reaction in the second vessel 1142, which may be independent of the first reaction). For example, in some embodiments, a first vessel 1141 is constructed and arranged for conducting a first reaction (e.g., a first part of a PCR reaction) and a second vessel 1142 is constructed and arranged for conducting a second reaction (e.g., a second part of a PCR reaction), which may be independent of the first reaction.

In FIGS. 7A and 7B the vessels are shown as having a one-to-one relationship with cassettes. However, as shown in FIG. 7C, the cassette 1120 of cartridge 1203 may comprise a set of two or more vessels 1140. In some embodiments, a cassette and/or a set of vessels includes at least 2, at least 4, at least 6, at least 8, at least 10, or at least 15 vessels. In certain embodiments, a cassette and/or a set of vessels includes less than or equal to 20 vessels, less than or equal to 15 vessels, less than or equal to 10 vessels, less than or equal to 8 vessels, less than or equal to 6 vessels, or less than or equal to 4 vessels. Combinations of the above-referenced ranges are also possible (e.g., at least 2 and less than or equal to 4 vessels). Other ranges are also possible. In some embodiments, vessels are located in a process body cassette. In some embodiments, the process body cassette is one piece. In some embodiments, the process body cassette comprises or consists of 18 vessels, e.g., arranged in two rows of 9. In some embodiments, a cartridge may further comprises a input/output cassette, a cassette for Solid Phase Reversible Immobilization (SPRI) cleanup (e.g., a Ampure XP cassette), and/or a primer cassette.

FIG. 7D shows a portion of another cartridge. A frame 1110 has openings to receive a cassette including multiple vessels. Fluid from one or more sources (not shown) is introduced into vessels 1140 through inlets 1185. Each vessel 1140 includes sidewalls 1145 and is covered by a cover 1190. Vent channels 1160 are positioned between the cover 1190 and a top surface 1191 joining the sidewalls (e.g., a top surface of the article forming the vessel), and allow gaseous fluid (e.g., displaced air) to pass from the vessel 1140 to an outlet (not shown). The cover 1190 has an extended portion 1192 having a depth 1193 (e.g., relative to top surface 1191) that can penetrate into the vessel 1140 and reduce the working volume of the vessel 1140. The depth of the extended portion can be modified to vary the working volume of the vessel. In some embodiments, different vessels (e.g., of a cassette) may be enclosed by a cover having extended portions having the same depth; however, in other embodiments, different vessels (e.g., of a cassette) may be enclosed by a cover having extended portions having different depths.

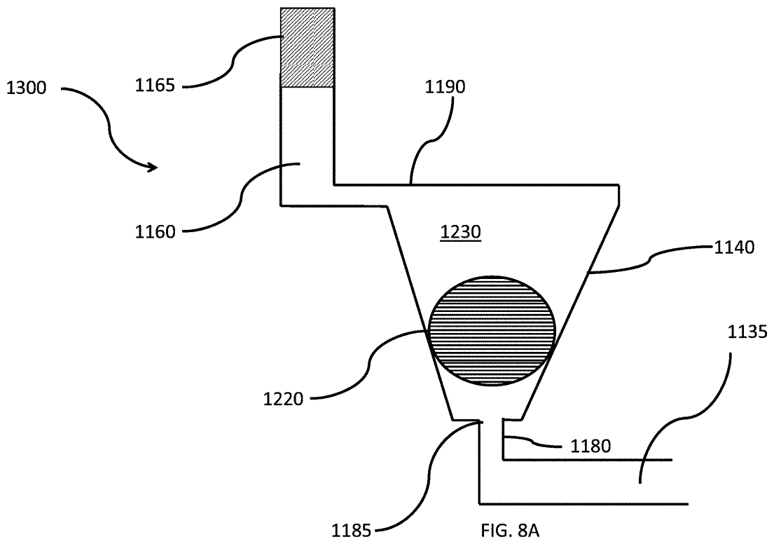

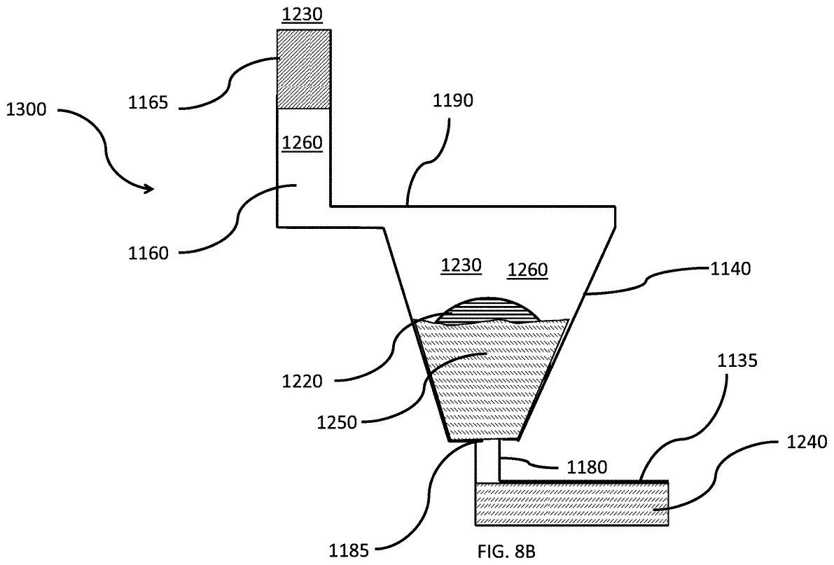

FIG. 8A shows a portion of a cartridge 1300, according to one or more embodiments, including a reagent 1220 inside vessel 1140. The cartridge 1300 is shown in a state prior to introduction of a sample fluid via microfluidic channel 1135. In one particular set of embodiments, reagent 1220 is dry. Also present in the vessel 1140 is an amount of air 1230, which occupies the volume of the vessel 1140 not occupied by reagent 1220.

According to some embodiments, the reagent may comprise one or more lyospheres with one or more reagents for performing one or more steps of a reaction (e.g., a reaction in a PCR process). It should be appreciated that while a spherical reagent is shown illustratively in FIG. 8A, in other embodiments, the reagent may have other shapes and/or configurations. For instance, in some embodiments, the reagent may be a fluid (e.g., a liquid). In some embodiments, the stored liquid reagent includes a primer, a buffer, a wash reagent, and/or an alcohol. In certain embodiments, the stored reagent is a stored dried reagent. Generally, in some embodiments, at least one of the cassettes and/or vessels (or set of vessels) contains a reagent, such as a stored reagent. In certain embodiments, the stored reagent may be used for conducting a reaction, and in some cases may be a reactant. In some embodiments, the stored reagent may be for conducting a PCR reaction.

In some embodiments, at least one of the cassettes and/or vessel (or set of vessels) contains one or more stored lyospheres. That is, in certain embodiments, the stored reagent is a stored lyosphere. For example, in one embodiment, at least one cassette and/or at least one vessel contains a single lyosphere. In another embodiment, at least one cassette and/or at least one vessel contains two or more lyospheres (e.g., two or more, three or more, or four or more lyospheres). In yet another embodiment, at least one cassette and/or at least one vessel contains a set of lyospheres. In some embodiments, at least a portion of the set of vessels contains at least one lyosphere disposed therein.

In some embodiments, a cartridge comprises a first cassette comprising a first set of vessels containing stored lyospheres and a second module comprising a second set of vessels containing stored lyospheres. In some such embodiments, the first and second cassettes are not be in fluid communication with one another (e.g., prior to, or after, insertion of the cassettes in the cartridge/frame, and/or during storage). As described above, in some embodiments, the vessel(s) containing a stored reagent (e.g., liquid reagent) is/are sealed so as to reduce or prevent evaporation of the stored reagent, and/or to reduce or prevent contamination of the stored reagent.

In an exemplary embodiment, a cartridge comprises a first cassette comprising a first set of vessels, a second cassette comprising a second set of vessels, a first set of stored reagents for conducting a first reaction (e.g., a first PCR reaction) contained in the first set of vessels, and a second set of stored reagents for conducting a second reaction (e.g., a second PCR reaction) contained in the second set of vessels. In some such embodiments, as described above, the cartridge may be constructed and arranged to allow first and second reactions to be performed in parallel. In certain embodiments, the cartridge may be constructed and arranged to allow fluid communication between the channel system and at least one of the first and second cassettes during the first and/or second reactions, respectively. As described in more detail below, the channel system may include first and second sets of channels. The first set of channels may be in fluid communication with the first cassette comprising the first set of vessels, and the second set of channels may be in fluid communication with the second cassette comprising the second set of vessels. The first and second set of channels may be in fluid communication with one another via one or more valves.

FIG. 8B shows a portion of cartridge 1300, according to one or more embodiments after a fluid (e.g., a sample fluid) 1240 containing at least one reagent has been introduced into vessel 1140. After fluid 1240 is introduced to the vessel via microfluidic channel 1135, orifice 1180, and inlet 1185, the fluid 1240 mixes with reagent 1220 (a portion of which is shown in FIG. 8A) already present in the vessel. In some embodiments, the reagent is at least partially dissolved in the liquid and reacts with the sample fluid 1240 to form a product fluid 1250. In other embodiments, reactions may take place in other forms. For instance, a reaction may take place on a surface of a substrate, in a solution, or in other configurations.

In some embodiments, the reaction may be facilitated by heating the vessel, as described in more detail below. At least some portion of the product is in vaporous form 1260. Furthermore, some amount of air 1230 or other gas (e.g., inert gas) may be present in the vessel 1140. At least a portion of the gaseous fluids (e.g., air and vapor) present in the vessel may be displaced to the vent channel 1160. The selective gas-permeable membrane 1165 functions to substantially prevent vapor 1260 from exiting cartridge 1300, while allowing air 1230 to exit the cartridge, thereby preserving a greater amount of fluid and/or reactant than if vapor 1260 were able to escape the cartridge.

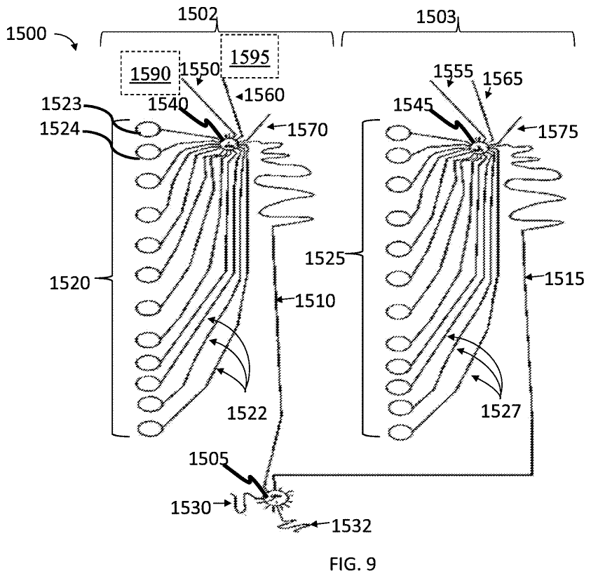

While the figures described above show only one or two vessels and one or two fluid sources (e.g., sample wells) for the sake of simplicity, more complex fluidic systems are contemplated and within the scope of the disclosure. For example, in one set of embodiments, as shown illustratively in FIG. 9, a channel system 1500 includes a first set of channels 1502 and a second set of channels 1503. The first set of channels may be used for conducting a first set of reactions (e.g., a first PCR reaction) and the second set of channels may be used for conducting a second set of reactions (e.g., a second PCR reaction). The first set of channels 1502 may include a first set of vessel channels 1522 connected to a first set of vessels 1520; and the second set of channels 1503 may include a second set of vessel channels 1527 connected to a second set of vessels 1525.

In some embodiments, the first set of vessels 1520 comprises a plurality of vessels (and vessel channels connected to the vessels), each vessel channel extending from valve a 1540. The valve 1540 may be connected to a common microfluidic channel 1510, which may be used for introducing reagents/fluids into, and/or removing reagents/fluids from, the channel system 1502. In certain embodiments, the second set of vessels 1525 comprises a plurality of vessels (and vessel channels connected to the vessels), each vessel extending from a valve 1545. The valve 1545 may be connected to a common microfluidic channel 1515, which may be used for introducing reagents fluids into, and/or removing reagents/fluids from, the channel system 1503.

As described herein, the vessels may be part of a cassette that is inserted or otherwise a part of a cartridge. For example, in one set of embodiments, first set of vessels 1520 (including, e.g., vessels 1523, 1524, etc.) shown in FIG. 9 may be a part of cassette 1121 (as shown in FIG. 7B), and second set of vessels 1525 may be a part of cassette 1122 (as shown in FIG. 7B). Channel system 1130 (FIG. 7B) may include channel system 1500 of FIG. 9. For example, channel 1135 (FIG. 7B) may be one of vessel channels 1522 (FIG. 9), and channel 1136 (FIG. 7B) may be one of vessel channels 1527 (FIG. 9). Other configurations are also possible.

Various configurations of channels and valves may be possible in a channel system described herein. For instance, in some embodiments a channel system comprises a valve 1505 and common microfluidic channels 1510 and 1515 extending from valve 1505. As shown illustratively in FIG. 9, the first and second sets of vessel channels may be separated from one another by at least one valve and/or by at least one common microfluidic channel. That is, in some embodiments, one or more common microfluidic channels may be positioned between a first set of vessel channels and a second set of vessel channels. In certain embodiments, a common microfluidic channel may be positioned between a first valve and a second valve. For example, common microfluidic channel 1510 is shown illustratively in FIG. 9 as being positioned between valve 1505 and valve 1540. In certain embodiments, common microfluidic channel 1515 is positioned between valve 1505 and valve 1545.

In some embodiments, the channel system comprises secondary channels such as a waste channel connected to a waste vessel, a sample inlet channel connected to a sample well, and/or an output channel connected to an output well. Referring again to FIG. 9, in some embodiments, valve 1540 may be connected to sample inlet channel 1550, output channel 1560, and/or waste channel 1570. The sample inlet channel may be connected to one or more sample wells (e.g., as part of a sample cassette 1590, in fluidic communication with sample inlet channel 1550). The output channel may be connected to one or more output wells (e.g., as part of a output cassette 1595, in fluidic communication with output channel 1560). The waste channel may be connected to one or more waste wells (e.g., as part of a waste cassette, not shown). In certain embodiments, valve 1545 may be connected to sample inlet channel 1555, output channel 1565, and/or waste channel 1575. The sample inlet channel may be connected to one or more sample wells (e.g., as part of the sample cassette, not shown). The output channel may be connected to one or more output wells (e.g., as part of the output cassette, not shown). The waste channel may be connected to one or more waste wells (e.g., as part of a waste cassette, not shown). In certain embodiments, valve 1505 is connected to one or more fluid inlet channels (e.g., fluid inlet channels 1530 and 1532) that may transport one or more fluids/reagents to the channel systems.

In some embodiments, the channel system comprises one or more channels (or set of channels) for conducting one or more reactions (e.g., a first PCR reaction, a second PCR reaction, etc.). For example, referring again to FIG. 9, in some embodiments, the first set of channels 1502 including first set of vessel channels 1522 connected to the first set of vessels 1520 are configured for conducting a first reaction, and the second set of channels 1503 including second set of vessel channels 1527 connected to the second set of vessels 1525 are configured for conducting a second reaction. The first and second reactions may be conducted in parallel, or sequentially.

In general, the channel systems or portions thereof described herein may be used to control the direction and/or volume of a fluid. The methods described herein may be useful for, for example, mixing and/or reacting two or more reagents and/or fluids in controlled volumes. In some embodiments, the order of the mixing and/or reactions may be controlled (e.g., by controlling and/or alternating the direction of fluid flow).

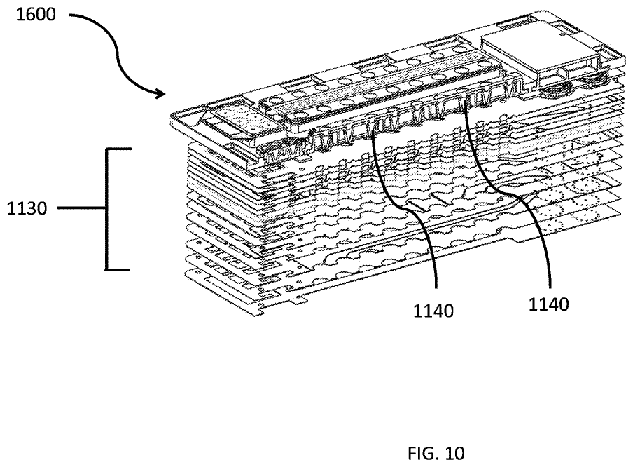

FIG. 10 shows an exploded view of a cartridge 1600 according to one or more embodiments. A set of layers 1130 may be used to form one or more fluidic channels and/or pathways in fluid communication with one or more vessels 1140. Upon use, the cartridge may be loaded into a bay as shown illustratively in FIG. 3. Cartridge 1600 may comprise the cartridge features described herein. The cartridge may comprise rows of vessels 1140. Each of the vessels may comprise the vessel features described herein.

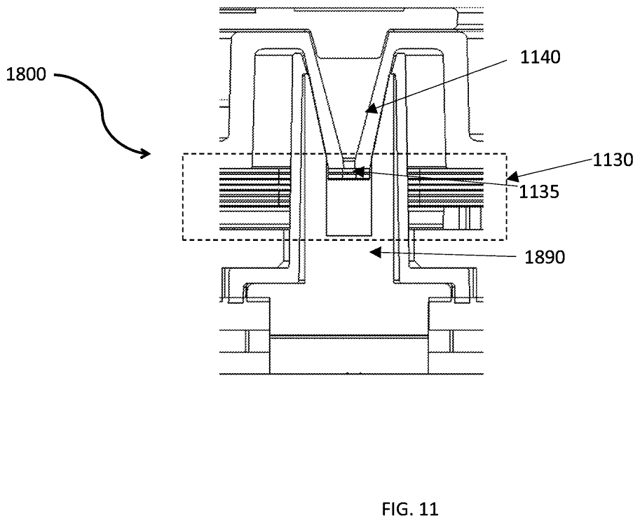

FIG. 11 shows a detailed view of a portion of a cartridge 1800, according to one or more embodiments. The cartridge comprises a microfluidic channel 1135 positioned in a channel system layer 1130 (e.g., a composite of several layers forming the channel system). A fluid is delivered from a source (not shown) to a vessel 1140 via the microfluidic channel 1135, where a desired reaction takes place. The cartridge 1800 further comprises a heated jacket 1890 surrounding at least a portion of the vessel 1140 to aid in changing (e.g., heating or cooling) a temperature in the vessel 1140 conducive to the desired reaction. Aspects of the heated jacket 1890 are discussed further below.

As described herein, a vessel, like the vessel 1140 shown in FIGS. 6A-8B, has an internal working volume. This volume may be defined, by the volume encompassed by the vessel sidewalls and the vessel cover. In some embodiments the internal volume of the vessel may be at least about 1 μL (e.g., volume of at least about 5 μL, at least about 10 μL, at least about 20 μL, at least about 30 μL, at least about 40 μL, at least about 50 μL, at least about 80 μL, at least about 100 μL, at least about 200 μL) and/or less than or equal to 500 μL (e.g., less than or equal to 400 μL, less than or equal to 300 μL, less than or equal to 200 μL, less than or equal to 100 μL, less than or equal to 80 μL, less than or equal to 60 μL, less than or equal to 40 μL, less than or equal to 20 μL). Combinations of the above-referenced ranges are also possible.

As described herein, the vessel may have any suitable shape. In some embodiments, at least one vessel has a conical shape. In certain embodiments, at least one vessel has a tapered cross-sectional shape defined by the sidewall(s), like that shown in, for example, FIG. 6A. The tapered shape may be substantially conical, like that of vessel 1140 shown in FIG. 6A, or may include some degree of curvature (e.g., the sidewall(s) may be/appear curved rather than straight from the perspective shown in FIG. 6A). The apex of the conical or tapered shape may be approximated as the inlet to the vessel. The vessel may have a taper angle defined as the angle formed between the axis and the surface (e.g., sidewall), as shown, for example, as θ in FIG. 6A. In some embodiments the taper angle of the vessel may be at least 5°, at least 10°, at least 20°, at least 30°, at least 40°, at least 45°, at least 50°, at least 60°, or at least 70°. In some embodiments the taper angle of the vessel may be less than or equal to 80°, less than or equal to 70°, less than or equal to 60°, less than or equal to 50°, less than or equal to 45°, less than or equal to 40°, less than or equal to 30°, less than or equal to 20°, or less than or equal to 10°. Combinations of the above-referenced ranges are also possible (e.g., at least 20° and less than or equal to 45°). Other ranges are also possible.

The shape of the vessel may be such that the vessel is free of a ledge (i.e., ledge free); such a configuration may facilitate mixing and/or reduce the presence of residue in the vessel. The shape of the vessel may facilitate the use of detection instruments (e.g., optical instruments) positioned adjacent (e.g., above, below) the vessel, so that, for example, the surface portions and/or fluid portions within the vessel receive an appropriate distribution of light used for a variety of purposes, including, for example, metrics, photochemistry, and process control.

In some embodiments, the vessel is configured to withstand a certain internal pressure for conducting a reaction in the vessel. For instance, the vessel may be configured to withstand a pressure of least 1 psi, at least 1.5 psi, at least 2 psi, at least 2.5 psi, at least 3 psi, or at least 3.5 psi,. The pressure in the vessel may be less than or equal to 4 psi, less than or equal to 3 psi, or less than or equal to 2 psi. Combinations of the above-referenced ranges are also possible (e.g., at least 1 psi and less than or equal to 4 psi). Combinations of the above-referenced ranges are also possible. A method of using a cartridge described herein may involve performing a reaction at one or more of the pressures described above in one or more vessels.

The vessel may be formed from any suitable material. According to certain embodiments the vessel is formed of a polymeric material. Non-limiting examples of polymeric materials include polypropylene, polyethylene, polystyrene, poly(acrylonitrile, butadiene, styrene), poly(styrene-co-acrylate), poly(methyl methacrylate), polycarbonate, polyester, poly(dimethylsiloxane), PVC, PTFE, PET, or blends of two or more such polymers. In some embodiments, a metal can be used. Non-limiting examples of metals include nickel, copper, stainless steel, bulk metallic glass, or other metals or alloys. In some embodiments, a ceramic may be used. Non-limiting examples of ceramics include glass, quartz, silica, alumina, zirconia, tungsten carbide, silicon carbide, or non-metallic materials such as graphite, silicon, or others. Other materials are also possible.

In some embodiments, a cartridge described herein may include different sets of vessels. The different sets of vessels may be contained in different cassettes. For example, in some embodiments, a first cassette comprises a first set of vessels and a second cassette comprises a second set of vessels. In certain embodiments, a third cassette may comprise a third set of vessels. Additional sets of vessels are also possible. As described herein, vessels may also be an integral part of a cartridge. In some embodiments, at least one set of vessels (e.g., as part of a cassette or a cartridge) includes at least 2, at least 4, at least 6, at least 8, at least 10, or at least 15 vessels. In certain embodiments, at least one set of vessels includes less than or equal to 20 vessels, less than or equal to 15 vessels, less than or equal to 10 vessels, less than or equal to 8 vessels, less than or equal to 6 vessels, or less than or equal to 4 vessels. Combinations of the above-referenced ranges are also possible (e.g., at least 2 and less than or equal to 4 vessels). Other ranges are also possible.

As shown in various figures, an orifice may be positioned between the microfluidic channel and the vessel proximate the inlet to the vessel. The orifice may have a constrictive flow path. The orifice may aid in keeping the sample in the vessel, for example, during pressure and temperature changes in the vessel.

The orifice may have a particular cross-sectional dimension (e.g., diameter). In some embodiments, a largest cross-sectional dimension of the orifice is smaller than a largest cross-sectional dimension of the microfluidic channel. In some embodiments, a largest cross-sectional dimension of the orifice is smaller than a largest cross-sectional dimension of the inlet of the vessel.

In some embodiments a dimension (e.g., average cross-sectional dimension, largest cross-sectional dimension, diameter, length) of the orifice may be at least 10 μm, at least 20 μm, at least 30 μm, at least 40 μm, at least 50 μm, at least 100 μm, at least 150 μm, at least 200 μm, at least 250 μm, at least 300 μm, at least 350 μm, at least 400 μm, at least 450 μm, at least 500 μm, at least 600 μm, or at least 700 μm. In certain embodiments, a dimension (e.g., average cross-sectional dimension, largest cross-sectional dimension diameter, length) of the orifice may be less than or equal to 1000 μm, less than or equal to 750 μm, less than or equal to 700 μm, less than or equal to 600 μm, less than or equal to 500 μm, less than or equal to 450 μm, less than or equal to 400 μm, less than or equal to 300 μm, less than or equal to 250 μm, less than or equal to 100 μm, less than or equal to 75 μm, less than or equal to 50 μm, or less than or equal to 25 μm. Combinations of the above-referenced ranges are also possible (e.g., at least 250 μm and less than or equal to 500 μm).

The orifice may be formed from any suitable material. According to certain embodiments, the orifice may be formed of a polymeric material. The material may comprise one or more of the materials listed herein for forming a vessel. The orifice may be formed in a layer positioned between a layer comprising the microfluidic channel and a layer forming the vessel.

As described herein, the cartridge may comprise a ventilation system that allows for selective gaseous components in the vessel to exit, while substantially maintaining other gaseous components. The ventilation system may comprise, for instance, a vent channel, a gas permeable membrane, and an outlet channel (shown in, for example, FIG. 6A). In some embodiments, the vent channel is defined, at least in part, by the vessel cover and sidewall of the vessel and/or a surface of an article forming the vessel. In some embodiments, the disclosed ventilation system allows for improved performance in the system, as unnecessary and/or undesired constituents (e.g., ambient air) may be evacuated from the vessel after a liquid stream (e.g., sample fluid) is introduced. Meanwhile, desired constituents (e.g., product vapor) remain in the vessel, where they may subsequently condense.

As described herein, a vent channel may be positioned between a vessel and a gas permeable membrane, which may be a part of a cover of the vessel. In some embodiments, the vent channel may be formed in a gap between the vessel cover and a sidewall of the vessel and/or a surface of an article forming the vessel. In some embodiments the vent channel may have a particular cross-sectional dimension (e.g., diameter). In some embodiments the cross-sectional dimension (e.g., largest cross-sectional dimension, average cross-sectional dimension) of the vent channel may be at least 50 μm, at least 100 μm, at least 150 μm, at least 200 μm, at least 250 μm, at least 300 μm, at least 350 μm, at least 400 μm, at least 450 μm, at least 500 μm, at least 600 μm, or at least 700 μm. In certain embodiments, the cross-sectional dimension (e.g., largest cross-sectional dimension, average cross-sectional dimension) of the vent channel may be less than or equal to 750 μm, less than or equal to 700 μm, less than or equal to 600 μm, less than or equal to 500 μm, less than or equal to 450 μm, less than or equal to 400 μm, less than or equal to 300 μm, less than or equal to 250 μm, or less than or equal to 100 μm. Combinations of the above-referenced ranges are also possible (e.g., at least 250 μm and less than or equal to 500 μm). Other ranges are also possible.

In some embodiments the vent channel may have a particular height and/or a width. In some embodiments the height and/or a width of the vent channel may be at least 50 μm, at least 100 μm, at least 150 μm, at least 200 μm, at least 250 μm, at least 300 μm, at least 350 μm, at least 400 μm, at least 450 μm, at least 500 μm, at least 600 μm, or at least 700 μm. In certain embodiments, the height and/or a width of the vent channel may be less than or equal to 750 μm, less than or equal to 700 μm, less than or equal to 600 μm, less than or equal to 500 μm, less than or equal to 450 μm, less than or equal to 400 μm, less than or equal to 300 μm, less than or equal to 250 μm, or less than or equal to 100 μm. Combinations of the above-referenced ranges are also possible (e.g., at least 250 μm and less than or equal to 500 μm). Other ranges are also possible.

In some embodiments the vent channel may have a particular length. In some embodiments the length of the vent channel may be at least 50 μm, at least 100 μm, at least 250 μm, at least 400 μm, at least 500 μm, at least 600 μm, at least 800 μm, at least 1 mm, at least 1 cm, or at least 1.5 cm. In certain embodiments, the length of the vent channel may be less than or equal to 2 cm, less than or equal to 1.5 cm, less than or equal to 1 cm, less than or equal to 800 μm, less than or equal to 600 μm, less than or equal to 500 μm, less than or equal to 400 μm, less than or equal to 250 μm, or less than or equal to 100 μm. Combinations of the above-referenced ranges are also possible (e.g., at least 250 μm and less than or equal to 500 μm). Other ranges are also possible.

As described herein, a cartridge may include a gas-permeable membrane. The gas permeable membrane may selectively allow certain gaseous constituents to pass while blocking passage of other gaseous or liquid components. The gas-permeable membrane may be configured to allow air to pass through the membrane while substantially preventing a liquid or vapor from passing across the membrane. In some embodiments, the gas-permeable membrane is positioned adjacent a vent channel (e.g., downstream of the vent channel) and/or an outlet of the vessel. The gas-permeable membrane may be configured as a part of a cover of a vessel in some embodiments, although other configurations are also possible.

The gas-permeable membrane may be formed from any suitable components. In some embodiments, the gas-permeable membrane may comprise a hydrophobic material. For example, the gas-permeable membrane may comprise PTFE, polyethylene, or any other suitable materials. In some embodiments, the gas-permeable membrane may comprise polycarbonate, silicone polymers, nylon, olefin polymers, polystyrene, acrylics, or other types of fluorocarbon polymers.

The gas-permeable membrane may have a particular average pore size. In some embodiments, the average pore size of the gas-permeable membrane may be at least 0.1 μm, at least 0.2 μm, at least 0.4 μm, at least 0.6 μm, or at least 0.8 μm. In some embodiments the average pore size may be less than or equal to 1 μm, less than or equal to 0.8 μm, less than or equal to 0.6 μm, less than or equal to 0.4 μm, or less than or equal to 0.2 μm. Combinations of the above-referenced ranges are also possible (e.g., at least 0.1 μm and less than or equal to 1 μm). In some embodiments, the \ average pore size of the membrane is in the range of 0.01 μm and 10 μm. Other ranges are also possible.

The gas-permeable membrane may have a particular thickness. In some embodiments, the thickness of the gas-permeable membrane may be at least 1 μm at least 50 μm, at least 100 μm, at least 200 μm, at least 300 μm, at least 400 μm, at least 500 μm, at least 600 μm, at least 800 μm. In some embodiments the thickness of the gas-permeable membrane may be less than or equal to 1 mm, less than or equal to 800 μm, less than or equal to 600 μm, less than or equal to 500 μm, less than or equal to 400 μm, less than or equal to 300 μm, less than or equal to 200 μm, less than or equal to 100 μm, less than or equal to 50 μm. Combinations of the above-referenced ranges are also possible (e.g., at least 1 μm and less than or equal to 1 mm). In some embodiments, membrane thickness is in the range of 0.01 mm and 5 mm. Other ranges are also possible.

The cartridge may further comprise an outlet channel positioned between the outlet and the vent channel. The outlet channel may be formed in a cover covering the cartridge.

The outlet channel may have any suitable dimensions. In some embodiments, the outlet channel has a cross-sectional dimension (e.g., a largest cross-sectional dimension, an average cross-sectional dimension) of at least 1 μm, at least 50 μm, at least 100 μm, at least 200 μm, at least 300 μm, at least 400 μm, at least 500 μm, at least 600 μm, at least 800 μm. In some embodiments the outlet channel has a cross-sectional dimension (e.g., a largest cross-sectional dimension, an average cross-sectional dimension) of less than or equal to 1 mm, less than or equal to 800 μm, less than or equal to 600 μm, less than or equal to 500 μm, less than or equal to 400 μm, less than or equal to 300 μm, less than or equal to 200 μm, less than or equal to 100 μm, or less than or equal to 50 μm. Combinations of the above-referenced ranges are also possible (e.g., at least 1 μm and less than or equal to 1 mm). In some embodiments, an outlet vent channel has cross-sectional dimensions of a minimum of 0.1 mm×0.1 mm and a maximum of 2.0 mm×2.0 mm. Other ranges are also possible.

The outlet channel may have any suitable length. In some embodiments, the outlet channel has a length of at least 1 mm, at least 2 mm, at least 4 mm, at least 6 mm, or at least 8 mm. In some embodiments, the outlet channel has a length of less than or equal to 1 cm, less than or equal to 8 mm, less than or equal to 6 mm, less than or equal to 4 mm, or less than or equal to 2 mm. Combinations of the above-referenced ranges are also possible (e.g., at least 1 mm and less than or equal to 1 cm). In some embodiments, an outlet vent channel has a length between a minimum 1.0 mm and a maximum of 100 mm. Other ranges are also possible.

In some cases, at least a portion of the one or more vessels may be sealed. In some embodiments, the cartridge may comprise a removable protective strip positioned adjacent the gas-permeable membrane, wherein the removable protective strip provides a fluid barrier. The protective strip may be a barrier to both gases and liquids. In some embodiments, the removable protective strip may be a foil or other fluid-impervious material that facilitates storage of a fluid and/or reagent in the vessel. For instance, in some embodiments in which the vessel is a part of a cassette, the vessel may contain a fluid and/or reagent such that, prior to insertion of the cassette into the frame or cartridge, the fluid does not substantially escape (e.g., by leaking, by evaporation) the cassette. Advantageously, sealing the vessels and/or cassettes may reduce or prevent evaporation of a stored liquid reagent and/or contamination of reagents. In some cases, upon use (e.g., insertion of the cassette into the frame or cartridge, or upon use of the cartridge), the protective strip may be removed by the user.

In some embodiments, the frame or cartridge includes one or more puncture components constructed and arranged to puncture one or more portions of the cassette upon insertion of the cassette into the frame or cartridge. In some such embodiments, upon insertion of the cassette into an opening of the frame, the puncturing component (e.g., located within or adjacent the opening into which the cassette is being inserted) punctures the cassette such that a reagent contained within the cassette is in fluidic communication with the channel system.

The two or more vessels may be in fluidic communication with the channel system such that fluids can be transported between each of the vessels. For example, after a first reaction (e.g., between a sample component and a first reagent positioned within the first vessel) takes place in a first vessel, the reaction product may be transported from the first vessel back to the channel system and into the second vessel for conducting a second reaction (e.g., between the reaction product and a second reagent). The same process can be used to transport fluids into various vessels for conducting various reactions (e.g., sequential reactions).

In some embodiments, the cartridge (or frame) comprises one or more puncture components constructed and arranged to puncture one or more portions of the vessel upon insertion of the vessel into the frame. In some such embodiments, upon insertion of the vessel into an opening of the cartridge or frame, the puncturing component (e.g., located within or adjacent the opening into which the cassette comprising the vessel is being inserted) punctures the vessel such that the vessel and/or a reagent contained within the vessel is in fluidic communication with the channel system. In some embodiments, a cartridge includes a series of puncture components, each puncture component aligned to puncture a seal of a vessel.

In certain embodiments, the vessels in the set of vessels (e.g., of a cassette) are not in fluid communication with each other prior to their insertion into the cartridge (e.g., prior to insertion of the cassette comprising the set of vessels into an opening of the cartridge and/or the frame). In some embodiments, a set of vessels contains two or more reagents stored therein, not in fluid communication with one another prior to insertion of the cassette including the vessels into the cartridge. For example, in some embodiments, a set of vessels comprises a first reagent stored in a first vessel therein and a second reagent stored in a second vessel therein, wherein the first and second reagents (and/or the first and second vessels) are not in fluid communication with one another prior to insertion of the cassette including the set of vessels into the cartridge. In certain embodiments, the set of vessels is inserted or fixed in the cartridge such that a first reagent and/or a second reagent stored therein are in fluid communication with the channel system (e.g., at least one channel of the channel system).

In some embodiments, a microfluidic channel (e.g., a common channel, a vessel channel) in fluid is in communication with the vessel and configured to deliver a fluid to the vessel. As shown in FIG. 6A, the microfluidic channel and the vessel may be in different planes as defined by the longest dimension of each. The system may also include other channels such as a vent channel, a waste channel, an inlet channel, as described herein.

In certain embodiments, one or more channels of the channel system has a particular cross-sectional dimension (e.g., average cross-sectional dimension, largest cross-sectional dimension). The “cross-sectional dimension” (e.g., a diameter) of the channel is measured perpendicular to the direction of fluid flow. In some embodiments, the cross-sectional dimension (e.g., average cross-sectional dimension, largest cross-sectional dimension) of the channel is less than or equal to about 2 mm, less than or equal to about 1 mm, less than or equal to about 800 microns, less than or equal to about 600 microns, less than or equal to about 500 microns, less than or equal to about 400 microns, or less than or equal to about 300 microns. In certain embodiments, the cross-sectional dimension (e.g., average cross-sectional dimension, largest cross-sectional dimension) of the channel is greater than or equal to about 250 microns, greater than or equal to about 300 microns, greater than or equal to about 400 microns, greater than or equal to about 500 microns, greater than or equal to about 600 microns, greater than or equal to about 800 microns, or greater than or equal to about 1 mm. Combinations of the above-referenced ranges are also possible (e.g., between about 250 microns and about 2 mm, between about 400 microns and about 1 mm, between about 300 microns and about 600 microns). Other ranges are also possible. In some cases, more than one channel or capillary may be used. “Microfluidic channels” refer to channels having an average cross-sectional dimension of less than 1 mm. In other embodiments, fluidic channels having a cross-sectional dimension (e.g., average cross-sectional dimension, largest cross-sectional dimension) greater than 1 mm are also possible.

One or more microfluidic channels of the channel system (e.g., a common channel, a vessel channel, a vent channel) may have any suitable internal volume. In some embodiments, the internal volume of the channel (e.g., microfluidic channel) may be at least 0.1 microliters, at least 0.5 microliters, at least 1 microliter, at least 2 microliters, at least 5 microliters, at least 7 microliters, at least 10 microliters, at least 12 microliters, at least 15 microliters, at least 20 microliters, at least 30 microliters, or at least 50 microliters. In certain embodiments, the internal volume of the microfluidic channel may be less than or equal to 100 microliters, less than or equal to 70 microliters, less than or equal to 50 microliters, less than or equal to 25 microliters, less than or equal to 10 microliters, or less than or equal to 5 microliters. Combinations of the above-referenced ranges are also possible (e.g., between 1 microliter and 10 microliters). Other ranges are also possible.

One or more channels (e.g., microfluidic channels) of the channel system can have any suitable cross-sectional shape (circular, oval, triangular, irregular, trapezoidal, square or rectangular, or the like). A microfluidic channel may also have an aspect ratio (length to average cross sectional dimension) of at least 2:1, more typically at least 3:1, at least 5:1, or at least 10:1 or more. A fluid (e.g., a sample) within the channel may partially or completely fill the channel.

In some embodiments, the one or more channels (e.g., microfluidic channels) may have a particular configuration. In certain embodiments, at least a portion of one or more microfluidic channels may be substantially linear in the direction of fluid flow. In some embodiments, substantially all of one or more microfluidic channels is substantially linear in the direction of fluid flow. In some embodiments, at least a portion of one or more microfluidic channels (e.g., a common microfluidic channel) may be curved, bent, serpentine, staggered, zig-zag, spiral, or combinations thereof. Advantageously, the use of a non-linear microfluidic channel (e.g., a serpentine common channel) permits an increased holding volume of the channel per unit length of the cartridge measured in the average direction of fluid flow as compared to a linear microfluidic channel.

Various methods for transporting fluids are possible in the systems described herein. In some embodiments, at least one vessel may be configured to receive a fluid (e.g., a fluid containing a sample, a reaction fluid, a waste fluid, etc.), such as to receive a fluid from the channel system. In certain embodiments, a reaction may be performed in a vessel. For example, the vessel may contain a first reagent (e.g., a first stored reagent) and the first reagent is reacted with a fluid to form a second fluid. In some cases, the reaction may be a chemical and/or biological reaction.

In some embodiments, at least one vessel may be configured to deliver a fluid (e.g., a fluid containing a sample, a reaction fluid, etc.), such as to deliver a fluid to the channel system. In some cases, at least one of the vessels may be refillable. For example, at least one vessel may be used to perform a reaction (e.g., between a stored reagent therein and a sample) and the vessel may be refilled with a new reagent after the first reaction is completed. In some such embodiments, the at least one vessel may be used to perform two or more reactions.

Referring back to FIG. 9, in some cases, at least a portion of a fluid transferred to a first vessel (e.g., vessel 1523) may be reacted with a first stored reagent present in the first vessel. In some such embodiments, at least a portion of the reacted fluid (e.g., after reacting with the first stored reagent) may be transferred to the common microfluidic channel (e.g., channel 1510) and subsequently transferred to the second vessel (e.g., vessel 1524), such that the portion of the fluid may react with a second stored reagent present in the second vessel. In some cases, upon entering the first vessel, the fluid may be exposed to a first reagent. In certain embodiments, upon entering the second vessel, the fluid is exposed to a second reagent. In some embodiments, a sample and/or reactant present in the fluid reacts with the first reagent and/or the second reagent.

In some embodiments, a second fluid (e.g., a reactant, a sample) may be transferred from the microfluidic channel to the second vessel via the second vessel channel. For example, at least a portion of a first fluid transferred to the first vessel may (or may not) be reacted with a first stored reagent present in the first vessel. In some cases, at least a portion of the first fluid may be transferred to a waste channel (e.g., connected to a waste cassette) such that at least a portion of the first fluid (e.g., the first reacted fluid) remains in the first vessel. A second fluid (e.g., a reactant, a sample) may be introduced into the common microfluidic channel (e.g., via the sample inlet channel, via a second valve connected to the common microfluidic channel) and at least a portion of the second fluid may be flowed to the first vessel such that the first fluid and the second fluid mix and/or react. In some such embodiments, at least a portion of the fluid (e.g., after mixing and/or reacting the first and second fluids) may be transferred to the common microfluidic channel and subsequently transferred to the second vessel, such that the portion of the fluid may react with a second stored reagent present in the second vessel. Third, fourth, fifth, sixth, seventh, eighth, etc. reactions may be performed in different vessels (e.g., third, fourth, fifth, sixth, seventh, eighth, etc. vessels) in a similar manner as described herein. Advantageously, numerous reaction and mixing steps may be facilitated through the use of a common microfluidic channel and a set of vessels. In some embodiments, the fluid may be transferred (e.g., flowed) to the common microfluidic channel and subsequently transferred to the output channel (e.g., the output channel connected to one or more output wells).

In some embodiments, at least one or more vessels (or set of vessels) contains one or more stored reagents. In certain embodiments, the stored reagent may be used for conducting a reaction, and in some cases may be a reactant. In some embodiments, the stored reagent may be for conducting a PCR reaction. In some embodiments, the stored reagent is a stored liquid reagent. In some embodiments, the stored liquid reagent includes a primer, a buffer, a wash reagent, and/or an alcohol.

In some embodiments, the stored reagent is a stored lyosphere. For example, in one embodiment, at least one vessel contains a single lyosphere. In another embodiment, at least one vessel contains two or more lyospheres (e.g., two or more, three or more, or four or more lyospheres). In yet another embodiment, at least one vessel contains a set of lyospheres. In some embodiments, at least a portion of the set of vessels contains at least one lyosphere disposed therein.

While a single lyosphere is shown in FIG. 8A, those skilled in the art would understand that one, or two or more, lyospheres may be present in each vessel in other embodiments. In some embodiments, the vessel(s) containing a stored reagent (e.g., liquid reagent) is/are sealed so as to reduce or prevent evaporation of the stored reagent, and/or to reduce or prevent contamination of the stored reagent.

In an exemplary embodiment, a cartridge comprises a first set of vessels, a second a second set of vessels, a first set of stored reagents for conducting a first reaction (e.g., a first PCR reaction) contained in the first set of vessels, and a second set of stored reagents for conducting a second reaction (e.g., a second PCR reaction) contained in the second set of vessels. In some such embodiments, as described above, the cartridge may be constructed and arranged to allow conduction of the first and second PCR reactions in parallel. In certain embodiments, the cartridge may be constructed and arranged to allow fluid communication between the channel system and at least one of the first and second cassettes during conduction of the first and/or second reactions, respectively. The channel system may include first and second sets of channels. The first set of channels may be in fluid communication with the first set of vessels, and the second set of channels may be in fluid communication with the second set of vessels. The first and second set of channels may be in fluid communication with one another via one or more valves.

In some cases, one or more cassettes and/or vessels are configured to receive a fluid such that a reaction may take place within the cassette(s) and/or vessel(s). For example, in some embodiments, one or more cassettes and/or vessels may receive a reactant and a fluid (e.g., a sample) such that the reactant and fluid/sample react within the one or more cassettes and/or vessels. In some embodiments, one or more cassettes and/or vessels comprise a reagent (i.e., a reagent cassette), a primer (i.e., a primer cassette), or a buffer (i.e., a buffer cassette). For example, in some cases, the reactant cassette (which may include one or more vessels) contains one or more lyospheres, as described herein. Reactants, reagents, primers, and buffers are also described in more detail herein.

In some embodiments, a cassette and/or a vessel may include one or more fluids and/or reagents for a particular reaction or analysis, or may be configured to receive one or more fluids and/or reagents for a particular reaction or analysis (e.g., a waste cassette, a reagent cassette, a primer cassette, a buffer cassette, a sample cassettes, an output cassette). In some cases, the one or more fluids and/or reagents may be present in one or more vessels of the cassette (e.g., during storage, during use). The cassette may be inserted into the cartridge by a user, in some cases. The cartridge may be configured such that the opening of the frame allows fluidic communication between the cassette and the channel system (e.g., a channel, port, or other fluidic component of the channel system).

As described herein, in some embodiments a fluid collection device may be used to collect a fluid sample from a subject or a patient. A “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A patient may be a subject diagnosed with a certain disease or bodily condition or otherwise known to have a disease or bodily condition. In some embodiments, a patient may be diagnosed as, or known to be, at risk of developing a disease or bodily condition. In other embodiments, a patient may be suspected of having or developing a disease or bodily condition, e.g., based on various clinical factors and/or other data.

In some embodiments, at least one of the cassettes and/or at least one set of vessels is constructed and arranged to be heated (or cooled). In embodiments comprising two or more cassettes, a first and second cassette (or first and second set of vessels) may be constructed and arranged to be heated (or cooled) independently. For example, in some embodiments, a temperature control device is configured to apply a first temperature to a first cassette and a second temperature to a second cassette (e.g., simultaneously or sequentially).

In some embodiments, the cartridge may comprise a temperature-control device. In certain embodiments, the cartridge may be in communication with the temperature-control device. In some embodiments, the cartridge comprises a lid (e.g., a heated lid) that can be temperature-controlled. For example, in some embodiments, the cartridge comprises a lid including a temperature control device. In some embodiments, the lid covers at least one vessel. In certain embodiments, the lid (e.g., the temperature-controlled lid) forms a top portion of a vessel. Lids that can be temperature-controlled may be, in some cases, translucent or transparent. Advantageously, the temperature-controllable lid may be configured to allow optical measurements to be taken therethrough.

In some embodiments, the temperature control device comprises one or more thermal pads, thermoelectric components, and/or thermistors. Those skilled in the art would be capable of selecting suitable temperature control devices based upon the teachings of this specification.

### Lid/Cover Temperature Control

In some embodiments, the cartridge further comprises a lid that can be temperature-controlled. The lid may covers the vessels (e.g., form a top portion of the vessel). In some embodiments, the lid is translucent or transparent. The lid may be configured to allow optical measurements to be taken therethrough.

In some embodiments, the cartridge is in communication with a temperature control device. The temperature control device may be configured to apply a first temperature to the lid. The temperature control device may comprises one or more thermal pads, thermoelectric components, and/or thermistors. In some cases, the temperature control device comprises a lid (e.g., heated lid).

### Heated Jackets

In some embodiments, a vessel or set of vessels is constructed and arranged to be heated by a heated thermal jacket that surrounds at least portion of the sidewalls of the vessel.

As described herein and in reference to the figures, according to certain embodiments, the cartridge may be used to carry out one or more chemical reactions (e.g., reactions related to a polymerase chain reaction process).

In one set of embodiments, a method may comprise flowing a first fluid comprising a first reagent (e.g., a sample fluid) from a source through the microfluidic channel. The first fluid may be a liquid. The first reagent may comprise, for example, a compound in the sample material. The microfluidic channel system may deliver fluid to vessels in series along one of the microfluidic channel paths or in parallel along multiple microfluidic channel paths. The system may include one or more sources of sample flowed to one or more vessels.

The method may comprise introducing at least a portion of the first fluid to a vessel having an internal working volume and containing a second reagent to fill a portion, but not all, of the internal working volume of the vessel with the first fluid. The internal working volume of the vessel may be a volume defined by the sidewalls and cover of the vessel. The second reagent may be a dry reagent prior to introduction of the first fluid. In some embodiments the second reagent may comprise a lyosphere. The first fluid may be introduced into an inlet of the vessel positioned at a bottom portion of the vessel. The step of introducing may further comprise passing the first fluid through a narrow orifice that is positioned proximate an inlet to the vessel. In some embodiments, a specific volume of fluid may be introduced to the vessel. The volume may be, for example, at least 5 μl, at least 10 μl, at least 20 μl, at least 30 μl, at least 40 μl, at least 50 μl, or at least 60 μl. The volume may be less than or equal to 70 μl, less than or equal to 60 μl, less than or equal to 50 μl, less than or equal to 40 μl, less than or equal to 30 μl, less than or equal to 20 μl, or less than or equal to 10 μl. Combinations of the above-referenced ranges are also possible (e.g., at least 5 μl and less than or equal to 70 μl). Other ranges are also possible.

In some embodiments a second fluid may be introduced to the vessel. The second fluid may be a hydrophobic fluid (e.g., an oil). The second fluid may be less dense than the first fluid and form a layer above the first fluid. The second fluid may function as a barrier preventing evaporation of the first fluid. In some embodiments a specific volume of the second fluid may be introduced. The volume may be, for example, at least 1 μl, at least 5 μl, at least 10 μl, at least 20 μl, at least 30 μl, at least 40 μl, at least 50 μl, or at least 60 μl. The volume may be less than or equal to 70 μl, less than or equal to 60 μl, less than or equal to 50 μl, less than or equal to 40 μl, less than or equal to 30 μl, less than or equal to 20 μl, or less than or equal to 10 μl. Combinations of the above-referenced ranges are also possible (e.g., at least 5 μl and less than or equal to 70 μl). Examples of hydrophobic fluids include, but are not limited to, mineral oil, fluorinated species, hydrocarbon-based solvents, paraffin beads, silicone oil, etc.

The method may comprise reacting (e.g., chemically or biologically) the first reagent with the second reagent, wherein during the reaction a ratio by volume of a liquid portion to a gaseous portion in the reaction vessel is at least 1 to 5 and less than or equal to 5 to 1. The liquid portion may comprise the first fluid which was delivered via the microfluidic channel and a reagent either present in liquid form in the vessel (e.g., previously stored in the vessel or transported into the vessel) or that has been dissolved in the vessel. The gaseous portion may comprise air present in the vessel, and any gaseous or vaporous products from the fluid introduced to the vessel, fluid previously present in the vessel, or fluid product resulting from the reaction of reagents.

In some embodiments, there may be a specific ratio by volume of a liquid portion to a gaseous portion in the reaction vessel, during the reaction. In some embodiments, the ratio of liquid to gas by volume may be at least 1 to 5, at least 2 to 5, at least 3 to 5, at least 4 to 5, at least 1 to 1, at least 5 to 4, at least 5 to 3, or at least 5 to 2. In some embodiments, the ratio of liquid to gas may be equal to or less than 5 to 1, equal to or less than 5 to 2, equal to or less than 5 to 3, equal to or less than 5 to 4, equal to or less than 1 to 1, equal to or less than 4 to 5, equal to or less than 3 to 5, or equal to or less than 2 to 5. Combinations of the above-referenced ranges are also possible (e.g., at least 1 to 5 and less than or equal to 5 to 1). Other ranges are also possible.

In some embodiments the chemical and/or biological reaction may be performed in the vessel within a specific pressure range. The pressure in the vessel may be, for example, at least 1 psi, at least 1.5 psi, at least 2 psi, at least 2.5 psi, at least 3 psi, or at least 3.5 psi,. The pressure in the vessel may be less than or equal to 4 psi, less than or equal to 3 psi, or less than or equal to 2 psi. Combinations of the above-referenced ranges are also possible (e.g., at least 1 psi and less than or equal to 4 psi). Other ranges are also possible.

The method may comprise allowing a portion of the gas (e.g., air) to pass through a vent channel in fluid communication with the vessel. For example, the method may comprise allowing air to pass through a membrane (e.g., a gas-permeable membrane) in fluid communication with the vessel while substantially preventing a liquid or vapor from passing across the membrane. Over the course of the reaction, vapor is generally formed. This vapor in conjunction with air already present in the vessel could cause undesired pressure build-up in the vessel. At the same time, venting the vapor could cause the loss of desired reaction product. According to certain embodiments, a selective gas permeable membrane allows for air to vent while substantially blocking the exit of vapor, thereby avoiding these two problems. At least a portion of gaseous vapor in the vessel may condense over the period of time it is within the vessel. In some embodiments, the membrane aids in maintaining a certain pressure range within the vessel during the reaction.

The vessel may be heated during at least a portion of the time in which liquid is within it. The vessel may be heated, for example, by a lid above the vessel. In some embodiments, the vessel may be heated to a specific temperature or range of temperatures. The temperature may be at least 4 degrees C., at least 10 degrees C., at least 20 degrees C., at least 30 degrees C., at least 40 degrees C., at least 50 degrees C., at least 60 degrees C., at least 70 degrees C., at least 80 degrees C., or at least 90 degrees C. The temperature may be less than or equal to 100 degrees C., less than or equal to 95 degrees C., less than or equal to 90 degrees C., less than or equal to 80 degrees C., less than or equal to 70 degrees C., less than or equal to 60 degrees C., less than or equal to 40 degrees C., or less than or equal to 20 degrees C. Combinations of the above-referenced ranges are also possible (e.g., at least 4 degrees C. and less than or equal to 95 degrees C.). Other ranges are also possible.

### Amplification (AMP) Methods

Described herein are methods of determining the nucleotide sequence contiguous to a known target nucleotide sequence. The methods may be implemented in an automated fashion using the systems disclosed herein. Traditional sequencing methods generate sequence information randomly (e.g., “shotgun” sequencing) or between two known sequences which are used to design primers. In contrast, certain of the methods described herein, in some embodiments, allow for determining the nucleotide sequence (e.g., sequencing) upstream or downstream of a single region of known sequence with a high level of specificity and sensitivity.

In some embodiments, the systems provided herein may be configured to implement, e.g., in an automated fashion, a method of enriching specific nucleotide sequences prior to determining the nucleotide sequence using a next-generation sequencing technology. In some embodiments, methods provided herein can relate to enriching samples comprising deoxyribonucleic acid (DNA). In some embodiments, methods provided herein comprise: (a) ligating a target nucleic acid comprising the known target nucleotide sequence with a universal oligonucleotide tail-adapter; (b) amplifying a portion of the target nucleic acid and the amplification strand of the universal oligonucleotide tail-adapter with a first adapter primer and a first target-specific primer; (c) amplifying a portion of the amplicon resulting from step (b) with a second adapter primer and a second target-specific primer; and (d) transferring the DNA solution to a user. In some embodiments, one or more steps of the methods may be performed within different vessels of a cartridge provided herein. In some embodiments, microfluidic channels and valves in the cartridge facilitate the transfer of reaction material/fluid from one vessel to another in the cartridge to permit reactions to proceed in an automated fashion. In some embodiments, a DNA solution can subsequently be sequenced with a first and second sequencing primer using a next-generation sequencing technology.

In some embodiments, a sample processed using a system provided herein comprises genomic DNA. In some embodiments, samples comprising genomic DNA include a fragmentation step preceding step (a). In some embodiments, each ligation and amplification step can optionally comprise a subsequent purification step (e.g., sample purification between step (a) and step (b), sample purification between step (b) and step (c), and/or sample purification following step (c)). For example, the method of enriching samples comprising genomic DNA can comprise: (a) fragmentation of genomic DNA; (b) ligating a target nucleic acid comprising the known target nucleotide sequence with a universal oligonucleotide tail-adapter; (c) post-ligation sample purification; (d) amplifying a portion of the target nucleic acid and the amplification strand of the universal oligonucleotide tail-adapter with a first adapter primer and a first target-specific primer; (e) post-amplification sample purification; (f) amplifying a portion of the amplicon resulting from step (d) with a second adapter primer and a second target-specific primer; (g) post-amplification sample purification; and (h) transferring the purified DNA solution to a user. In some embodiments, steps of the methods may be performed within different vessels of a cartridge provided herein. In some embodiments, microfluidic channels and valves in the cartridge facilitate the transfer of reaction material/fluid from one vessel to another in the cartridge in an automated fashion. In The purified sample can subsequently be sequenced with a first and second sequencing primer using a next-generation sequencing technology.

In some embodiments, systems and methods provided herein may be used for processing nucleic acids as depicted in the exemplary workflow in FIG. 1. A nucleic acid sample 120 is provided. In some embodiments, the sample comprises RNA. In some embodiments, the sample comprises DNA (e.g., double-stranded complementary DNA (cDNA) and/or double-stranded genomic DNA (gDNA) 102). In some embodiments, the nucleic acid sample is subjected to a step 102 comprising nucleic acid end repair and/or dA tailing. In some embodiments, the nucleic acid sample is subjected to a step 104 comprising adapter ligation. In some embodiments, a universal oligonucleotide adapter 122 is ligated to one or more nucleic acids in the nucleic acid sample. In some embodiments, the ligation step comprises blunt-end ligation. In some embodiments, the ligation step comprises sticky-end ligation. In some embodiments, the ligation step comprises overhang ligation. In some embodiments, the ligation step comprises TA ligation. In some embodiments, the dA tailing step 102 is performed to generate an overhang in the nucleic acid sample that is complementary to an overhang in the universal oligonucleotide adapter (e.g., TA ligation). In some embodiments, a universal oligonucleotide adapter is ligated to both ends of one or more nucleic acids in the nucleic acid sample to generate a nucleic acid 124 flanked by universal oligonucleotide adapters. In some embodiments, an initial round of amplification is performed using an adapter primer 130 and a first target-specific primer 132. In some embodiments, the amplified sample is subjected to a second round of amplification using an adapter primer and a second target-specific primer 134. In some embodiments, the second target-specific primer is nested relative to the first target-specific primer. In some embodiments, the second target-specific primer comprises additional sequences 5′ to a hybridization sequence (e.g., common sequence) that may include barcode, index, adapter sequences, or sequencing primer sites. In some embodiments, the second target-specific primer is further contacted by an additional primer that hybridizes with the common sequence of the second target-specific primer, as depicted by 134. In some embodiments, the second round of amplification generates a nucleic acid 126 that is suitable for nucleic acid sequencing (e.g., next generation sequencing methods).

In some embodiments, systems and methods provided herein may be used for processing nucleic acids as described in PCT International Application No. PCT/US2017/051924, which was filed on Sep. 15, 2017, and which claims priority under 35 U.S.C. § 119(e) to U.S. Provisional Patent Application No. 62/395,339, which was filed on Sep. 15, 2016, and in PCT International Application No. PCT/US2017/051927, which was filed on Sep. 15, 2017, and which claims priority under 35 U.S.C. § 119(e) to U.S. Provisional Patent Application No. 62/395,347, which was filed on Sep. 15, 2016, the entire contents of each of which relating to nucleic acid library preparation are hereby incorporated by reference.

In some embodiments, a sample processed using a system provided herein comprises ribonucleic acid (RNA). In some embodiments, a system provided herein can be useful for processing RNA by a method comprising: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) amplifying a portion of the target nucleic acid and the amplification strand of the universal oligonucleotide tail-adapter with a first adapter primer and a first target-specific primer; (h) amplifying a portion of the amplicon resulting from step (g) with a second adapter primer and a second target-specific primer; and (i) transferring the cDNA solution to a user. In some embodiments, one or more steps of the methods may be performed within different vessels of a cartridge provided herein. In some embodiments, cDNA solution can subsequently be sequenced with a first and second sequencing primer using a next-generation sequencing technology.

In some embodiments, each ligation and amplification step can optionally comprise a subsequent sample purification step (e.g., sample purification step between step (f) and step (g), sample purification step between step (g) and step (h), and/or sample purification following step (h)). For example, the method of enriching samples comprising RNA can comprise: (a) contacting a target nucleic acid molecule comprising the known target nucleotide sequence with a population of random primers under hybridization conditions; (b) performing a template-dependent extension reaction that is primed by a hybridized random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) subjecting the nucleic acid to end-repair, phosphorylation, and adenylation; (f) ligating the target nucleic acid comprising the known target nucleotide sequence with a universal oligonucleotide tail-adapter; (g) post-ligation sample purification; (h) amplifying a portion of the target nucleic acid and the amplification strand of the universal oligonucleotide tail-adapter with a first adapter primer and a first target-specific primer; (i) post-amplification sample purification; (j) amplifying a portion of the amplicon resulting from step (h) with a second adapter primer and a second target-specific primer; (k) post-amplification sample purification; and (1) transferring the purified cDNA solution to a user. In some embodiments, one or more steps of the methods may be performed within different vessels of a cartridge provided herein. The purified sample can subsequently be sequenced with a first and second sequencing primer using a next-generation sequencing technology.

In some embodiments, the systems provided herein may be configured to implement, e.g., in an automated fashion, a method of enriching nucleotide sequences that comprise a known target nucleotide sequence downstream from an adjacent region of unknown nucleotide sequence (e.g., nucleotide sequences comprising a 5′ region comprising an unknown sequence and a 3′ region comprising a known sequence). In some embodiments, the method comprises: (a) contacting a target nucleic acid molecule comprising the known target nucleotide sequence with an initial target-specific primer under hybridization conditions; (b) performing a template-dependent extension reaction that is primed by a hybridized initial target-specific primer and that uses the target nucleic acid molecule as a template; (c) contacting the product of step (b) with a population of tailed random primers under hybridization conditions; (d) performing a template-dependent extension reaction that is primed by a hybridized tailed random primer and that uses the portion of the target nucleic acid molecule downstream of the site of hybridization as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (e), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein.

In some embodiments, the systems provided herein may be configured to implement, e.g., in an automated fashion, a method of enriching nucleotide sequences that comprise a known target nucleotide sequence upstream from an adjacent region of unknown nucleotide sequence (e.g., nucleotide sequences comprising a 5′ region comprising a known sequence and a 3′ region comprising an unknown sequence). In some embodiments, the method comprises: (a) contacting a target nucleic acid molecule comprising the known target nucleotide sequence with a population of tailed random primers under hybridization conditions; (b) performing a template-dependent extension reaction that is primed by a hybridized tailed random primer and that uses the portion of the target nucleic acid molecule downstream of the site of hybridization as a template; (c) contacting the product of step (b) with an initial target-specific primer under hybridization conditions; (d) performing a template-dependent extension reaction that is primed by a hybridized initial target-specific primer and that uses the target nucleic acid molecule as a template; (e) amplifying a portion of the target nucleic acid molecule and the tailed random primer sequence with a first tail primer and a first target-specific primer; (f) amplifying a portion of the amplicon resulting from step (e) with a second tail primer and a second target-specific primer; and (g) transferring the cDNA solution to a user. The cDNA solution can subsequently be sequenced with a first and second sequencing primer using a next-generation sequencing technology. In some embodiments, the population of tailed random primers comprises single-stranded oligonucleotide molecules having a 5′ nucleic acid sequence identical to a first sequencing primer and a 3′ nucleic acid sequence comprising from about 6 to about 12 random nucleotides. In some embodiments, the first target-specific primer comprises a nucleic acid sequence that can specifically anneal to the known target nucleotide sequence of the target nucleic acid at the annealing temperature. In some embodiments, the second target-specific primer comprises a 3′ portion comprising a nucleic acid sequence that can specifically anneal to a portion of the known target nucleotide sequence comprised by the amplicon resulting from step (c), and a 5′ portion comprising a nucleic acid sequence that is identical to a second sequencing primer and the second target-specific primer is nested with respect to the first target-specific primer. In some embodiments, the first tail primer comprises a nucleic acid sequence identical to the tailed random primer. In some embodiments, the second tail primer comprises a nucleic acid sequence identical to a portion of the first sequencing primer and is nested with respect to the first tail primer. In some embodiments, one or more steps of the method may be performed within different vessels of a cartridge provided herein. In some embodiments, the method further involves a step of contacting the sample with RNase after extension of the initial target-specific primer. In some embodiments, the tailed random primer can form a hair-pin loop structure. In some embodiments, the initial target-specific primer and the first target-specific primer are identical. In some embodiments, the tailed random primer further comprises a barcode portion comprising 6-12 random nucleotides between the 5′ nucleic acid sequence identical to a first sequencing primer and the 3′ nucleic acid sequence comprising 6-12 random nucleotides.

### Universal Oligonucleotide Tail Adapter

As used herein, the term “universal oligonucleotide tail-adapter” refers to a nucleic acid molecule comprised of two strands (a blocking strand and an amplification strand) and comprising a first ligatable duplex end and a second unpaired end. The blocking strand of the universal oligonucleotide tail-adapter comprises a 5′ duplex portion. The amplification strand comprises an unpaired 5′ portion, a 3′ duplex portion, a 3′ T overhang, and nucleic acid sequences identical to a first and second sequencing primer. The duplex portions of the blocking strand and the amplification strand are substantially complementary and form the first ligatable duplex end comprising a 3′ T overhang and the duplex portion is of sufficient length to remain in duplex form at the ligation temperature.

In some embodiments, the portion of the amplification strand that comprises a nucleic acid sequence identical to a first and second sequencing primer can be comprised, at least in part, by the 5′ unpaired portion of the amplification strand.

In some embodiments, the universal oligonucleotide tail-adapter can comprise a duplex portion and an unpaired portion, wherein the unpaired portion comprises only the 5′ portion of the amplification strand, i.e., the entirety of the blocking strand is a duplex portion.

In some embodiments, the universal oligonucleotide tail-adapter can have a “Y” shape, i.e., the unpaired portion can comprise portions of both the blocking strand and the amplification strand which are unpaired. The unpaired portion of the blocking strand can be shorter than, longer than, or equal in length to the unpaired portion of the amplification strand. In some embodiments, the unpaired portion of the blocking strand can be shorter than the unpaired portion of the amplification strand. Y shaped universal oligonucleotide tail-adapters have the advantage that the unpaired portion of the blocking strand will not be subject to 3′ extension during a PCR regimen. In some embodiments, the blocking strand of the universal oligonucleotide tail-adapter can further comprise a 3′ unpaired portion which is not substantially complementary to the 5′ unpaired portion of the amplification strand; and wherein the 3′ unpaired portion of the blocking strand is not substantially complementary to or substantially identical to any of the primers. In some embodiments, the blocking strand of the universal oligonucleotide tail-adapter can further comprise a 3′ unpaired portion which will not specifically anneal to the 5′ unpaired portion of the amplification strand at the annealing temperature; and wherein the 3′ unpaired portion of the blocking strand will not specifically anneal to any of the primers or the complements thereof at the annealing temperature.

### First Amplification Step

As used herein, the term “first target-specific primer” refers to a single-stranded oligonucleotide comprising a nucleic acid sequence that can specifically anneal under suitable annealing conditions to a nucleic acid template that has a strand characteristic of a target nucleic acid.

In some embodiments, a primer (e.g., a target specific primer) can comprise a 5′ tag sequence portion. In some embodiments, multiple primers (e.g., all first-target specific primers) present in a reaction can comprise identical 5′ tag sequence portions. In some embodiments, in a multiplex PCR reaction, different primer species can interact with each other in an off-target manner, leading to primer extension and subsequently amplification by DNA polymerase. In such embodiments, these primer dimers tend to be short, and their efficient amplification can overtake the reaction and dominate resulting in poor amplification of desired target sequence. Accordingly, in some embodiments, the inclusion of a 5′ tag sequence in primers (e.g., on target specific primer(s)) may result in formation of primer dimers that contain the same complementary tails on both ends. In some embodiments, in subsequent amplification cycles, such primer dimers would denature into single-stranded DNA primer dimers, each comprising complementary sequences on their two ends which are introduced by the 5′ tag. In some embodiments, instead of primer annealing to these single stranded DNA primer dimers, an intra-molecular hairpin (a panhandle like structure) formation may occur due to the proximate accessibility of the complementary tags on the same primer dimer molecule instead of an inter-molecular interaction with new primers on separate molecules. Accordingly, in some embodiments, these primer dimers may be inefficiently amplified, such that primers are not exponentially consumed by the dimers for amplification; rather the tagged primers can remain in high and sufficient concentration for desired specific amplification of target sequences. In some embodiments, accumulation of primer dimers may be undesirable in the context of multiplex amplification because they compete for and consume other reagents in the reaction.

In some embodiments, a 5′ tag sequence can be a GC-rich sequence. In some embodiments, a 5′ tag sequence may comprise at least 50% GC content, at least 55% GC content, at least 60% GC content, at least 65% GC content, at least 70% GC content, at least 75% GC content, at least 80% GC content, or higher GC content. In some embodiments, a tag sequence may comprise at least 60% GC content. In some embodiments, a tag sequence may comprise at least 65% GC content.

As used herein, the term “first adapter primer” refers to a nucleic acid molecule comprising a nucleic acid sequence identical to a 5′ portion of the first sequencing primer. As the first tail-adapter primer is therefore identical to at least a portion of the sequence of the amplification strand (as opposed to complementary), it will not be able to specifically anneal to any portion of the universal oligonucleotide tail-adapter itself.

In the first PCR amplification cycle of the first amplification step, the first target-specific primer can specifically anneal to a template strand of any nucleic acid comprising the known target nucleotide sequence. Depending upon the orientation with which the first target-specific primer was designed, a sequence upstream or downstream of the known target nucleotide sequence will be synthesized as a strand complementary to the template strand. If, during the extension phase of PCR, the 5′ end of the template strand terminates in a ligated universal oligonucleotide tail-adapter, the 3′ end of the newly synthesized product strand will comprise sequence complementary to the first tail-adapter primer. In subsequent PCR amplification cycles, both the first target-specific primer and the first tail-adapter primer will be able to specifically anneal to the appropriate strands of the target nucleic acid sequence and the sequence between the known nucleotide target sequence and the universal oligonucleotide tail-adapter can be amplified (i.e., copied).

### Second Amplification Step

As used herein, the term “second target-specific primer” refers to a single-stranded oligonucleotide comprising a 3′ portion comprising a nucleic acid sequence that can specifically anneal to a portion of the known target nucleotide sequence comprised by the amplicon resulting from a preceding amplification step, and a 5′ portion comprising a nucleic acid sequence that is identical to a second sequencing primer. The second target-specific primer can be further contacted by an additional primer (e.g., a primer having 3′ sequencing adapter/index sequences) that hybridizes with the common sequence of the second target-specific primer. In some embodiments, the additional primer may comprise additional sequences 5′ to the hybridization sequence that may include barcode, index, adapter sequences, or sequencing primer sites. In some embodiments, the additional primer is a generic sequencing adapter/index primer. The second target-specific primer is nested with respect to the first target-specific primer. In some embodiments, the second target-specific primer is nested with respect to the first target-specific primer by at least 3 nucleotides, e.g., by 3 or more, 4 or more, 5 or more, 6 or more, 7 or more, 8 or more, 9 or more, 10 or more, or 15 or more nucleotides.

In some embodiments, all of the second target-specific primers present in a reaction comprise the same 5′ portion. In some embodiments, the 5′ portion of the second target-specific primers can serve to suppress primer dimers as described for the 5′ tag of the first target-specific primer described above herein.

In some embodiments, the first and second target-specific primers are substantially complementary to the same strand of the target nucleic acid. In some embodiments, the portions of the first and second target-specific primers that specifically anneal to the known target sequence can comprise a total of at least 20 unique bases of the known target nucleotide sequence, e.g., 20 or more unique bases, 25 or more unique bases, 30 or more unique bases, 35 or more unique bases, 40 or more unique bases, or 50 or more unique bases. In some embodiments, the portions of the first and second target-specific primers that specifically anneal to the known target sequence can comprise a total of at least 30 unique bases of the known target nucleotide sequence.

As used herein, the term “second adapter primer” refers to a nucleic acid molecule comprising a nucleic acid sequence identical to a portion of the first sequencing primer and is nested with respect to the first adapter primer. As the second tail-adapter primer is therefore identical to at least a portion of the sequence of the amplification strand (as opposed to complementary), it will not be able to specifically anneal to any portion of the universal oligonucleotide tail-adapter itself. In some embodiments, the second adapter primer is identical to the first sequencing primer.

The second adapter primer should be nested with respect to the first adapter primer, that is, the first adapter primer comprises a nucleic acid sequence identical to the amplification strand which is not comprised by the second adapter primer and which is located closer to the 5′ end of the amplification primer than any of the sequence identical to the amplification strand which is comprised by the second adapter primer. In some embodiments, the second adapter primer is nested by at least 3 nucleotides, e.g., by 3 nucleotides, by 4 nucleotides, by 5 nucleotides, by 6 nucleotides, by 7 nucleotides, by 8 nucleotides, by 9 nucleotides, by 10 nucleotides or more.

In some embodiments, the first adapter primer can comprise a nucleic acid sequence identical to about the 20 5′-most bases of the amplification strand of the universal oligonucleotide tail-adapter and the second adapter primer can comprise a nucleic acid sequence identical to about 30 bases of the amplification strand of the universal oligonucleotide tail-adapter, with a 5′ base which is at least 3 nucleotides 3′ of the 5′ terminus of the amplification strand.

In some embodiments, nested primer sets may be used. In some embodiments, the use of nested adapter primers eliminates the possibility of producing final amplicons that are amplifiable (e.g., during bridge PCR or emulsion PCR) but cannot be efficiently sequenced using certain techniques. In some embodiments, hemi-nested primer sets may be used.

### Sample Purification Step

In some embodiments, target nucleic acids and/or amplification products thereof can be isolated from enzymes, primers, or buffer components before and/or after any appropriate step of a method. Any suitable methods for isolating nucleic acids may be used. In some embodiments, the isolation can comprise Solid Phase Reversible Immobilization (SPRI) cleanup. Methods for SPRI cleanup are well known in the art, e.g., Agencourt AMPure XP-PCR Purification (Cat No. A63880, Beckman Coulter; Brea, Calif.). In some embodiments, enzymes can be inactivated by heat treatment.

In some embodiments, unhybridized primers can be removed from a nucleic acid preparation using appropriate methods (e.g., purification, digestion, etc.). In some embodiments, a nuclease (e.g., exonuclease I) is used to remove primer from a preparation. In some embodiments, such nucleases are heat inactivated subsequent to primer digestion. Once the nucleases are inactivated, a further set of primers may be added together with other appropriate components (e.g., enzymes, buffers) to perform a further amplification reaction.

### Sequencing

In some aspects, the technology described herein relates to methods of enriching nucleic acid samples for oligonucleotide sequencing. In some embodiments, the sequencing can be performed by a next-generation sequencing method. As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/ Roche Diagnostics); solid-phase, reversible dye-terminator sequencing (Solexa/Illumina); SOLiD technology (Applied Biosystems); Ion semiconductor sequencing (ION Torrent); DNA nanoball sequencing (Complete Genomics); and technologies available from Pacific Biosciences, Intelligen Bio-systems, and Oxford Nanopore Technologies. In some embodiments, the sequencing primers can comprise portions compatible with the selected next-generation sequencing method. Next-generation sequencing technologies and the constraints and design parameters of associated sequencing primers are well known in the art (see, e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; Zhang et al., “The impact of next-generation sequencing on genomics”, J Genet Genomics, 2011, 38(3):95-109; (Nyren, P. et al. Anal Biochem 208: 17175 (1993); Bentley, D. R. Curr Opin Genet Dev 16:545-52 (2006); Strausberg, R. L., et al. Drug Disc Today 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; and 20070070349; which are incorporated by reference herein in their entireties).

In some embodiments, the sequencing step relies upon the use of a first and second sequencing primer. In some embodiments, the first and second sequencing primers are selected to be compatible with a next-generation sequencing method as described herein.

Methods of aligning sequencing reads to known sequence databases of genomic and/or cDNA sequences are well known in the art, and software is commercially available for this process. In some embodiments, reads (less the sequencing primer and/or adapter nucleotide sequence) which do not map, in their entirety, to wild-type sequence databases can be genomic rearrangements or large indel mutations. In some embodiments, reads (less the sequencing primer and/or adapter nucleotide sequence) comprising sequences which map to multiple locations in the genome can be genomic rearrangements.

### AMP Primers

In some embodiments, the four types of primers (first and second target-specific primers and first and second adapter primers) are designed such that they will specifically anneal to their complementary sequences at an annealing temperature of from about 61 to 72° C., e.g., from about 61 to 69° C., from about 63 to 69° C., from about 63 to 67° C., from about 64 to 66° C. In some embodiments, the four types of primers are designed such that they will specifically anneal to their complementary sequences at an annealing temperature of less than 72° C. In some embodiments, the four types of primers are designed such that they will specifically anneal to their complementary sequences at an annealing temperature of less than 70° C. In some embodiments, the four types of primers are designed such that they will specifically anneal to their complementary sequences at an annealing temperature of less than 68° C. In some embodiments, the four types of primers are designed such that they will specifically anneal to their complementary sequences at an annealing temperature of about 65° C. In some embodiments, systems provided herein are configured to alter vessel temperature (e.g., by cycling between different temperature ranges) to facilitate primer annealing.

In some embodiments, the portions of the target-specific primers that specifically anneal to the known target nucleotide sequence will anneal specifically at a temperature of about 61 to 72° C., e.g., from about 61 to 69° C., from about 63 to 69° C., from about 63 to 67° C., from about 64 to 66° C. In some embodiments, the portions of the target-specific primers that specifically anneal to the known target nucleotide sequence will anneal specifically at a temperature of about 65° C. in a PCR buffer.

In some embodiments, the primers and/or adapters described herein cannot comprise modified bases (e.g., the primers and/or adapters cannot comprise a blocking 3′ amine).

### Nucleic Acid Extension, Amplification, and PCR

In some embodiments, methods described herein comprise an extension regimen or step. In such embodiments, extension may proceed from one or more hybridized tailed random primers, using the nucleic acid molecules which the primers are hybridized to as templates. Extension steps are described herein. In some embodiments, one or more tailed random primers can hybridize to substantially all of the nucleic acids in a sample, many of which may not comprise a known target nucleotide sequence. Accordingly, in some embodiments, extension of random primers may occur due to hybridization with templates that do not comprise a known target nucleotide sequence.

In some embodiments, methods described herein may involve a polymerase chain reaction (PCR) amplification regimen, involving one or more amplification cycles. Amplification steps of the methods described herein can each comprise a PCR amplification regimen, i.e., a set of polymerase chain reaction (PCR) amplification cycles. In some embodiments, systems provided herein are configured to alter vessel temperature (e.g., by cycling between different temperature ranges) to facilitate different PCR steps, e.g., melting, annealing, elongation, etc.

In some embodiments, system provided herein are configured to implement an amplification regimen in an automated fashion. As used herein, the term “amplification regimen” refers to a process of specifically amplifying (increasing the abundance of) a nucleic acid of interest. In some embodiments, exponential amplification occurs when products of a previous polymerase extension serve as templates for successive rounds of extension. In some embodiments, a PCR amplification regimen according to methods disclosed herein may comprise at least one, and in some cases at least 5 or more iterative cycles. In some embodiments, each iterative cycle comprises steps of: 1) strand separation (e.g., thermal denaturation); 2) oligonucleotide primer annealing to template molecules; and 3) nucleic acid polymerase extension of the annealed primers. In should be appreciated that any suitable conditions and times involved in each of these steps may be used. In some embodiments, conditions and times selected may depend on the length, sequence content, melting temperature, secondary structural features, or other factors relating to the nucleic acid template and/or primers used in the reaction. In some embodiments, an amplification regimen according to methods described herein is performed in a thermal cycler, many of which are commercially available.

In some embodiments, a nucleic acid extension reaction involves the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and are commercially available. One group of nucleic acid polymerases are thermostable, i.e., they retain function after being subjected to temperatures sufficient to denature annealed strands of complementary nucleic acids, e.g., 94° C., or sometimes higher. A non-limiting example of a protocol for amplification involves using a polymerase (e.g., Phoenix Taq, VeraSeq) under the following conditions: 98° C. for 30 s, followed by 14-22 cycles comprising melting at 98° C. for 10 s, followed by annealing at 68° C. for 30 s, followed by extension at 72° C. for 3 min, followed by holding of the reaction at 4° C. However, other appropriate reaction conditions may be used. In some embodiments, annealing/extension temperatures may be adjusted to account for differences in salt concentration (e.g., 3° C. higher to higher salt concentrations). In some embodiments, slowing the ramp rate (e.g., 1° C./s, 0.5° C./s, 0.28° C./s, 0.1° C./s or slower), for example, from 98° C. to 65° C., improves primer performance and coverage uniformity in highly multiplexed samples. In some embodiments, systems provided herein are configured to alter vessel temperature (e.g., by cycling between different temperature ranges, having controlled ramp up or down rates) to facilitate amplification.

In some embodiments, a nucleic acid polymerase is used under conditions in which the enzyme performs a template-dependent extension. In some embodiments, the nucleic acid polymerase is DNA polymerase I, Taq polymerase, Phoenix Taq polymerase, Phusion polymerase, T4 polymerase, T7 polymerase, Klenow fragment, Klenow exo-, phi29 polymerase, AMV reverse transcriptase, M-MuLV reverse transcriptase, HIV-1 reverse transcriptase, VeraSeq ULtra polymerase, VeraSeq HF 2.0 polymerase, EnzScript, or another appropriate polymerase. In some embodiments, a nucleic acid polymerase is not a reverse transcriptase. In some embodiments, a nucleic acid polymerase acts on a DNA template. In some embodiments, the nucleic acid polymerase acts on an RNA template. In some embodiments, an extension reaction involves reverse transcription performed on an RNA to produce a complementary DNA molecule (RNA-dependent DNA polymerase activity). In some embodiments, a reverse transcriptase is a mouse moloney murine leukemia virus (M-MLV) polymerase, AMV reverse transcriptase, RSV reverse transcriptase, HIV-1 reverse transcriptase, HIV-2 reverse transcriptase, or another appropriate reverse transcriptase.

In some embodiments, a nucleic acid amplification reaction involves cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. In some embodiments, strand separation according to methods described herein is achieved by heating the nucleic acid sample above its melting temperature (Tₘ). In some embodiments, for a sample containing nucleic acid molecules in a reaction preparation suitable for a nucleic acid polymerase, heating to 94° C. is sufficient to achieve strand separation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), and a carrier (e.g., 0.01 to 0.5% BSA). A non-limiting example of a suitable buffer comprises 50 mM KCl, 10 mM Tris-HCl (pH 8.8 at 25° C.), 0.5 to 3 mM MgCl₂, and 0.1% BSA.

In some embodiments, a nucleic acid amplification involves annealing primers to nucleic acid templates having a strands characteristic of a target nucleic acid. In some embodiments, a strand of a target nucleic acid can serve as a template nucleic acid.

As used herein, the term “anneal” refers to the formation of one or more complementary base pairs between two nucleic acids. In some embodiments, annealing involves two complementary or substantially complementary nucleic acid strands hybridizing together. In some embodiments, in the context of an extension reaction, annealing involves the hybridization of primer to a template such that a primer extension substrate for a template-dependent polymerase enzyme is formed. In some embodiments, conditions for annealing (e.g., between a primer and nucleic acid template) may vary based of the length and sequence of a primer. In some embodiments, conditions for annealing are based upon a Tₘ (e.g., a calculated Tₘ) of a primer. In some embodiments, an annealing step of an extension regimen involves reducing the temperature following a strand separation step to a temperature based on the Tₘ (e.g., a calculated Tₘ) for a primer, for a time sufficient to permit such annealing. In some embodiments, a Tₘ can be determined using any of a number of algorithms (e.g., OLIGO™ (Molecular Biology Insights Inc. Colorado) primer design software and VENTRO NTI™ (Invitrogen, Inc. California) primer design software and programs available on the internet, including Primer3, Oligo Calculator, and NetPrimer (Premier Biosoft; Palo Alto, Calif.; and freely available on the world wide web (e.g., at premierbiosoft.com/netprimer/netprlaunch/Help/xnetprlaunch.html)). In some embodiments, the Tₘ of a primer can be calculated using the following formula, which is used by NetPrimer software and is described in more detail in Frieir, et al. PNAS 1986 83:9373-9377 which is incorporated by reference herein in its entirety.

Tₘ=ΔH/(ΔS+R*In(C/4))+16.6 log ([K⁺]/(1+0.7 [K⁺]))−273.15

wherein: ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). In some embodiments, the closer an annealing temperature is to the Tₘ, the more specific is the annealing. In some embodiments, the time used for primer annealing during an extension reaction (e.g., within the context of a PCR amplification regimen) is determined based, at least in part, upon the volume of the reaction (e.g., with larger volumes involving longer times). In some embodiments, the time used for primer annealing during an extension reaction (e.g., within the context of a PCR amplification regimen) is determined based, at least in part, upon primer and template concentrations (e.g., with higher relative concentrations of primer to template involving less time than lower relative concentrations). In some embodiments, depending upon volume and relative primer/template concentration, primer annealing steps in an extension reaction (e.g., within the context of an amplification regimen) can be in the range of 1 second to 5 minutes, 10 seconds to 2 minutes, or 30 seconds to 2 minutes. As used herein, “substantially anneal” refers to an extent to which complementary base pairs form between two nucleic acids that, when used in the context of a PCR amplification regimen, is sufficient to produce a detectable level of a specifically amplified product.

As used herein, the term “polymerase extension” refers to template-dependent addition of at least one complementary nucleotide, by a nucleic acid polymerase, to the 3′ end of a primer that is annealed to a nucleic acid template. In some embodiments, polymerase extension adds more than one nucleotide, e.g., up to and including nucleotides corresponding to the full length of the template. In some embodiments, conditions for polymerase extension are based, at least in part, on the identity of the polymerase used. In some embodiments, the temperature used for polymerase extension is based upon the known activity properties of the enzyme. In some embodiments, in which annealing temperatures are below the optimal temperatures for the enzyme, it may be acceptable to use a lower extension temperature. In some embodiments, enzymes may retain at least partial activity below their optimal extension temperatures. In some embodiments, a polymerase extension (e.g., performed with thermostable polymerases such as Taq polymerase and variants thereof) is performed at 65° C. to 75° C. or 68° C. to 72° C. In some embodiments, methods provided herein involve polymerase extension of primers that are annealed to nucleic acid templates at each cycle of a PCR amplification regimen. In some embodiments, a polymerase extension is performed using a polymerase that has relatively strong strand displacement activity. In some embodiments, polymerases having strong strand displacement are useful for preparing nucleic acids for purposes of detecting fusions (e.g., 5′ fusions).

In some embodiments, primer extension is performed under conditions that permit the extension of annealed oligonucleotide primers. As used herein, the term “conditions that permit the extension of an annealed oligonucleotide such that extension products are generated” refers to the set of conditions (e.g., temperature, salt and co-factor concentrations, pH, and enzyme concentration) under which a nucleic acid polymerase catalyzes primer extension. In some embodiments, such conditions are based, at least in part, on the nucleic acid polymerase being used. In some embodiments, a polymerase may perform a primer extension reaction in a suitable reaction preparation. In some embodiments, a suitable reaction preparation contains one or more salts (e.g., 1 to 100 mM KCl, 0.1 to 10 mM MgCl₂), at least one buffering agent (e.g., 1 to 20 mM Tris-HCl), a carrier (e.g., 0.01 to 0.5% BSA), and one or more NTPs (e.g, 10 to 200 μM of each of dATP, dTTP, dCTP, and dGTP). A non-limiting set of conditions is 50 mM KCl, 10 mM Tris-HCl (pH 8.8 at 25° C.), 0.5 to 3 mM MgCl₂, 200 μM each dNTP, and 0.1% BSA at 72° C., under which a polymerase (e.g., Taq polymerase) catalyzes primer extension. In some embodiments, conditions for initiation and extension may include the presence of one, two, three or four different deoxyribonucleoside triphosphates (e.g., selected from dATP, dTTP, dCTP, and dGTP) and a polymerization-inducing agent such as DNA polymerase or reverse transcriptase, in a suitable buffer. In some embodiments, a “buffer” may include solvents (e.g., aqueous solvents) plus appropriate cofactors and reagents which affect pH, ionic strength, etc.

In some embodiments, systems provided herein are configured to implement in an automated fashion multiple nucleic acid amplification cycles. In some embodiments, nucleic acid amplification involve up to 5, up to 10, up to 20, up to 30, up to 40 or more rounds (cycles) of amplification. In some embodiments, nucleic acid amplification may comprise a set of cycles of a PCR amplification regimen from 5 cycles to 20 cycles in length. In some embodiments, an amplification step may comprise a set of cycles of a PCR amplification regimen from 10 cycles to 20 cycles in length. In some embodiments, each amplification step can comprise a set of cycles of a PCR amplification regimen from 12 cycles to 16 cycles in length. In some embodiments, an annealing temperature can be less than 70° C. In some embodiments, an annealing temperature can be less than 72° C. In some embodiments, an annealing temperature can be about 65° C. In some embodiments, an annealing temperature can be from about 61 to about 72° C.

In various embodiments, methods and compositions described herein relate to performing a PCR amplification regimen with one or more of the types of primers described herein. As used herein, “primer” refers to an oligonucleotide capable of specifically annealing to a nucleic acid template and providing a 3′ end that serves as a substrate for a template-dependent polymerase to produce an extension product which is complementary to the template. In some embodiments, a primer is single-stranded, such that the primer and its complement can anneal to form two strands. Primers according to methods and compositions described herein may comprise a hybridization sequence (e.g., a sequence that anneals with a nucleic acid template) that is less than or equal to 300 nucleotides in length, e.g., less than or equal to 300, or 250, or 200, or 150, or 100, or 90, or 80, or 70, or 60, or 50, or 40, or 30 or fewer, or 20 or fewer, or 15 or fewer, but at least 6 nucleotides in length. In some embodiments, a hybridization sequence of a primer may be 6 to 50 nucleotides in length, 6 to 35 nucleotides in length, 6 to 20 nucleotides in length, 10 to 25 nucleotides in length.

Any suitable method may be used for synthesizing oligonucleotides and primers. In some embodiments, commercial sources offer oligonucleotide synthesis services suitable for providing primers for use in methods and compositions described herein (e.g., INVITROGEN™ Custom DNA Oligos (Life Technologies, Grand Island, N.Y.) or custom DNA Oligos from Integrated DNA Technologies (Coralville, Iowa)).

### DNA Shearing/Fragmentation

Nucleic acids used herein (e.g., prior to sequencing) can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a nucleic acid can be mechanically sheared by sonication. In some embodiments, systems provided here may have one or more vessels, e.g., within a cassette that is fitted within a cartridge, in which nucleic acids are sheared, e.g., mechanically or enzymatically.

In some embodiments, a target nucleic acid is not sheared or digested. In some embodiments, nucleic acid products of preparative steps (e.g., extension products, amplification products) are not sheared or enzymatically digested.

In some embodiments, when a target nucleic acid is RNA, the sample can be subjected to a reverse transcriptase regimen to generate a DNA template and the DNA template can then be sheared. In some embodiments, target RNA can be sheared before performing a reverse transcriptase regimen. In some embodiments, a sample comprising target RNA can be used in methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers.

### Target Nucleic Acid

As used herein, the term “target nucleic acid” refers to a nucleic acid molecule of interest (e.g., a nucleic acid to be analyzed). In some embodiments, a target nucleic acid comprises both a target nucleotide sequence (e.g., a known or predetermined nucleotide sequence) and an adjacent nucleotide sequence which is to be determined (which may be referred to as an unknown sequence). A target nucleic acid can be of any appropriate length. In some embodiments, a target nucleic acid is double-stranded. In some embodiments, the target nucleic acid is DNA. In some embodiments, the target nucleic acid is genomic or chromosomal DNA (gDNA). In some embodiments, the target nucleic acid can be complementary DNA (cDNA). In some embodiments, the target nucleic acid is single-stranded. In some embodiments, the target nucleic acid can be RNA (e.g., mRNA, rRNA, tRNA, long non-coding RNA, microRNA).

In some embodiments, the target nucleic acid can be comprised by genomic DNA. In some embodiments, the target nucleic acid can be comprised by ribonucleic acid (RNA), e.g., mRNA. In some embodiments, the target nucleic acid can be comprised by cDNA. Many of the sequencing methods suitable for use in the methods described herein provide sequencing runs with optimal read lengths of tens to hundreds of nucleotide bases (e.g., Ion Torrent technology can produce read lengths of 200-400 bp). Target nucleic acids comprised, for example, by genomic DNA or mRNA, can be comprised by nucleic acid molecules which are substantially longer than this optimal read length. In order for the amplified nucleic acid portion resulting from the second amplification step to be of a suitable length for use in a particular sequencing technology, the average distance between the known target nucleotide sequence and an end of the target nucleic acid to which the universal oligonucleotide tail-adapter can be ligated should be as close to the optimal read length of the selected technology as possible. For example, if the optimal read-length of a given sequencing technology is 200 bp, then the nucleic acid molecules amplified in accordance with the methods described herein should have an average length of about 400 bp or less. Target nucleic acids comprised by, e.g., genomic DNA or mRNA, can be sheared, e.g., mechanically or enzymatically sheared, to generate fragments of any desired size. Non-limiting examples of mechanical shearing processes include sonication, nebulization, and AFA™ shearing technology available from Covaris (Woburn, Mass.). In some embodiments, a target nucleic acid comprised by genomic DNA can be mechanically sheared by sonication.

In some embodiments, when the target nucleic acid is comprised by RNA, the sample can be subjected to a reverse transcriptase regimen to generate a DNA template and the DNA template can then be sheared. In some embodiments, target RNA can be sheared before performing the reverse transcriptase regimen. In some embodiments, a sample comprising target RNA can be used in the methods described herein using total nucleic acids extracted from either fresh or degraded specimens; without the need of genomic DNA removal for cDNA sequencing; without the need of ribosomal RNA depletion for cDNA sequencing; without the need of mechanical or enzymatic shearing in any of the steps; by subjecting the RNA for double-stranded cDNA synthesis using random hexamers; and by subjecting the nucleic acid to end-repair, phosphorylation, and adenylation.

In some embodiments, the known target nucleotide sequence can be comprised by a gene rearrangement. The methods described herein are suited for determining the presence and/or identity of a gene rearrangement as the identity of only one half of the gene rearrangement must be previously known (i.e., the half of the gene rearrangement which is to be targeted by the gene-specific primers). In some embodiments, the gene rearrangement can comprise an oncogene. In some embodiments, the gene rearrangement can comprise a fusion oncogene.

As used herein, the term “known target nucleotide sequence” refers to a portion of a target nucleic acid for which the sequence (e.g., the identity and order of the nucleotide bases of the nucleic acid) is known. For example, in some embodiments, a known target nucleotide sequence is a nucleotide sequence of a nucleic acid that is known or that has been determined in advance of an interrogation of an adjacent unknown sequence of the nucleic acid. A known target nucleotide sequence can be of any appropriate length.

In some embodiments, a target nucleotide sequence (e.g., a known target nucleotide sequence) has a length of 10 or more nucleotides, 30 or more nucleotides, 40 or more nucleotides, 50 or more nucleotides, 100 or more nucleotides, 200 or more nucleotides, 300 or more nucleotides, 400 or more nucleotides, 500 or more nucleotides. In some embodiments, a target nucleotide sequence (e.g., a known target nucleotide sequence) has a length in the range of 10 to 100 nucleotides, 10 to 500 nucleotides, 10 to 1000 nucleotides, 100 to 500 nucleotides, 100 to 1000 nucleotides, 500 to 1000 nucleotides, 500 to 5000 nucleotides.

In some embodiments, methods are provided herein for determining sequences of contiguous (or adjacent) portions of a nucleic acid. As used herein, the term “nucleotide sequence contiguous to” refers to a nucleotide sequence of a nucleic acid molecule (e.g., a target nucleic acid) that is immediately upstream or downstream of another nucleotide sequence (e.g., a known nucleotide sequence). In some embodiments, a nucleotide sequence contiguous to a known target nucleotide sequence may be of any appropriate length. In some embodiments, a nucleotide sequence contiguous to a known target nucleotide sequence comprises 1 kb or less of nucleotide sequence, e.g., 1 kb or less of nucleotide sequence, 750 bp or less of nucleotide sequence, 500 bp or less of nucleotide sequence, 400 bp or less of nucleotide sequence, 300 bp or less of nucleotide sequence, 200 bp or less of nucleotide sequence, 100 bp or less of nucleotide sequence. In some embodiments, in which a sample comprises different target nucleic acids comprising a known target nucleotide sequence (e.g., a cell in which a known target nucleotide sequence occurs multiple times in its genome, or on separate, non-identical chromosomes), there may be multiple sequences which comprise “a nucleotide sequence contiguous to” the known target nucleotide sequence. As used herein, the term “determining a (or the) nucleotide sequence,” refers to determining the identity and relative positions of the nucleotide bases of a nucleic acid.

In some embodiments, a known target nucleic acid can contain a fusion sequence resulting from a gene rearrangement. In some embodiments, methods described herein are suited for determining the presence and/or identity of a gene rearrangement. In some embodiments, the identity of one portion of a gene rearrangement is previously known (e.g., the portion of a gene rearrangement that is to be targeted by the gene-specific primers) and the sequence of the other portion may be determined using methods disclosed herein. In some embodiments, a gene rearrangement can involve an oncogene. In some embodiments, a gene rearrangement can comprise a fusion oncogene.

### Samples

In some embodiments, a target nucleic acid is present in or obtained from an appropriate sample (e.g., a food sample, environmental sample, biological sample e.g., blood sample, etc.). In some embodiments, the target nucleic acid is a biological sample obtained from a subject. In some embodiments a sample can be a diagnostic sample obtained from a subject. In some embodiments, a sample can further comprise proteins, cells, fluids, biological fluids, preservatives, and/or other substances. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample can be obtained from a subject in need of treatment for a disease associated with a genetic alteration, e.g., cancer or a hereditary disease. In some embodiments, a known target sequence is present in a disease-associated gene.

In some embodiments, a sample is obtained from a subject in need of treatment for cancer. In some embodiments, the sample comprises a population of tumor cells, e.g., at least one tumor cell. In some embodiments, the sample comprises a tumor biopsy, including but not limited to, untreated biopsy tissue or treated biopsy tissue (e.g., formalin-fixed and/or paraffin-embedded biopsy tissue).

In some embodiments, the sample is freshly collected. In some embodiments, the sample is stored prior to being used in methods and compositions described herein. In some embodiments, the sample is an untreated sample. As used herein, “untreated sample” refers to a biological sample that has not had any prior sample pre-treatment except for dilution and/or suspension in a solution. In some embodiments, a sample is obtained from a subject and preserved or processed prior to being utilized in methods and compositions described herein. By way of non-limiting example, a sample can be embedded in paraffin wax, refrigerated, or frozen. A frozen sample can be thawed before determining the presence of a nucleic acid according to methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g., anti-coagulant or nuclease inhibitor) and any combination thereof. In some embodiments, a sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in methods and compositions described herein. Suitable methods and processes for processing, preservation, or treatment of samples for nucleic acid analysis may be used in the method disclosed herein. In some embodiments, a sample can be a clarified fluid sample. In some embodiments, a sample can be clarified by low-speed centrifugation (e.g., 3,000×g or less) and collection of the supernatant comprising the clarified fluid sample.

In some embodiments, a nucleic acid present in a sample can be isolated, enriched, or purified prior to being utilized in methods and compositions described herein. Suitable methods of isolating, enriching, or purifying nucleic acids from a sample may be used. For example, kits for isolation of genomic DNA from various sample types are commercially available (e.g., Catalog Nos. 51104, 51304, 56504, and 56404; Qiagen; Germantown, Md.). In some embodiments, methods described herein relate to methods of enriching for target nucleic acids, e.g., prior to a sequencing of the target nucleic acids. In some embodiments, a sequence of one end of the target nucleic acid to be enriched is not known prior to sequencing. In some embodiments, methods described herein relate to methods of enriching specific nucleotide sequences prior to determining the nucleotide sequence using a next-generation sequencing technology. In some embodiments, methods of enriching specific nucleotide sequences do not comprise hybridization enrichment.

### Target Genes (ALK, ROS1, RET) and Therapeutic Applications

In some embodiments of methods described herein, a determination of the sequence contiguous to a known oligonucleotide target sequence can provide information relevant to treatment of disease. Thus, in some embodiments, methods disclosed herein can be used to aid in treating disease. In some embodiments, a sample can be from a subject in need of treatment for a disease associated with a genetic alteration. In some embodiments, a known target sequence is a sequence of a disease-associated gene, e.g., an oncogene. In some embodiments, a sequence contiguous to a known oligonucleotide target sequence and/or the known oligonucleotide target sequence can comprise a mutation or genetic abnormality which is disease-associated, e.g., a SNP, an insertion, a deletion, and/or a gene rearrangement. In some embodiments, a sequence contiguous to a known target sequence and/or a known target sequence present in a sample comprised sequence of a gene rearrangement product. In some embodiments, a gene rearrangement can be an oncogene, e.g., a fusion oncogene.

Certain treatments for cancer are particularly effective against tumors comprising certain oncogenes, e.g., a treatment agent which targets the action or expression of a given fusion oncogene can be effective against tumors comprising that fusion oncogene but not against tumors lacking the fusion oncogene. Methods described herein can facilitate a determination of specific sequences that reveal oncogene status (e.g., mutations, SNPs, and/or rearrangements). In some embodiments, methods described herein can further allow the determination of specific sequences when the sequence of a flanking region is known, e.g., methods described herein can determine the presence and identity of gene rearrangements involving known genes (e.g., oncogenes) in which the precise location and/or rearrangement partner are not known before methods described herein are performed.

In some embodiments, a subject is in need of treatment for lung cancer. In some embodiments, e.g., when the sample is obtained from a subject in need of treatment for lung cancer, the known target sequence can comprise a sequence from a gene selected from the group of ALK, ROS1, and RET. Accordingly, in some embodiments, gene rearrangements result in fusions involving the ALK, ROS1, or RET. Non-limiting examples of gene arrangements involving ALK, ROS1, or RET are described in, e.g., Soda et al. Nature 2007 448561-6: Rikova et al. Cell 2007 131:1190-1203; Kohno et al. Nature Medicine 2012 18:375-7; Takouchi et al. Nature Medicine 2012 18:378-81; which are incorporated by reference herein in their entireties. However, it should be appreciated that the precise location of a gene rearrangement and the identity of the second gene involved in the rearrangement may not be known in advance . Accordingly, in methods described herein, the presence and identity of such rearrangements can be detected without having to know the location of the rearrangement or the identity of the second gene involved in the gene rearrangement.

In some embodiments, the known target sequence can comprise sequence from a gene selected from the group of: ALK, ROS1, and RET.

In some embodiments, the presence of a gene rearrangement of ALK in a sample obtained from a tumor in a subject can indicate that the tumor is susceptible to treatment with a treatment selected from the group consisting of: an ALK inhibitor; crizotinib (PF-02341066); AP26113; LDK378; 3-39; AF802; IPI-504; ASP3026; AP-26113; X-396; GSK-1838705A; CH5424802; diamino and aminopyrimidine inhibitors of ALK kinase activity such as NVP-TAE684 and PF-02341066 (see, e.g., Galkin et al., Proc Natl Acad Sci USA, 2007, 104:270-275; Zou et al., Cancer Res, 2007, 67:4408-4417; Hallberg and Palmer F1000 Med Reports 2011 3:21; Sakamoto et al., Cancer Cell 2011 19:679-690; and molecules disclosed in WO 04/079326). All of the foregoing references are incorporated by reference herein in their entireties. An ALK inhibitor can include any agent that reduces the expression and/or kinase activity of ALK or a portion thereof, including, e.g., oligonucleotides, small molecules, and/or peptides that reduce the expression and/or activity of ALK or a portion thereof. As used herein “anaplastic lymphoma kinase” or “ALK” refers to a transmembrane tyROS line kinase typically involved in neuronal regulation in the wildtype form. The nucleotide sequence of the ALK gene and mRNA are known for a number of species, including human (e.g., as annotated under NCBI Gene ID: 238).

In some embodiments, the presence of a gene rearrangement of ROS1 in a sample obtained from a tumor in a subject can indicate that the tumor is susceptible to treatment with a treatment selected from the group consisting of: a ROS1 inhibitor and an ALK inhibitor as described herein above (e.g., crizotinib). A ROS1 inhibitor can include any agent that reduces the expression and/or kinase activity of ROS1 or a portion thereof, including, e.g., oligonucleotides, small molecules, and/or peptides that reduce the expression and/or activity of ROS1 or a portion thereof. As used herein “c-ros oncogene 1” or “ROS1” (also referred to in the art as ros-1) refers to a transmembrane tyrosine kinase of the sevenless subfamily and which interacts with PTPN6. Nucleotide sequences of the ROS1 gene and mRNA are known for a number of species, including human (e.g., as annotated under NCBI Gene ID: 6098).

In some embodiments, the presence of a gene rearrangement of RET in a sample obtained from a tumor in a subject can indicate that the tumor is susceptible to treatment with a treatment selected from the group consisting of: a RET inhibitor; DP-2490, DP-3636, SU5416; BAY 43-9006, BAY 73-4506 (regorafenib), ZD6474, NVP-AST487, sorafenib, RPI-1, XL184, vandetanib, sunitinib, imatinib, pazopanib, axitinib, motesanib, gefitinib, and withaferin A (see, e.g., Samadi et al., Surgery 2010 148:1228-36; Cuccuru et al., JNCI 2004 13:1006-1014; Akeno-Stuart et al., Cancer Research 2007 67:6956; Grazma et al., J Clin Oncol 2010 28:15s 5559; Mologni et al., J Mol Endocrinol 2006 37:199-212; Calmomagno et al., Journal NCI 2006 98:326-334; Mologni, Curr Med Chem 2011 18:162-175; and the compounds disclosed in WO 06/034833; U.S. Patent Publication 2011/0201598 and U.S. Pat. No. 8,067,434). All of the foregoing references are incorporated by reference herein in their entireties. A RET inhibitor can include any agent that reduces the expression and/or kinase activity of RET or a portion thereof, including, e.g., oligonucleotides, small molecules, and/or peptides that reduce the expression and/or activity of RET or a portion thereof. As used herein, “rearranged during transfection” or “RET” refers to a receptor tyrosine kinase of the cadherin superfamily which is involved in neural crest development and recognizes glial cell line-derived neurotrophic factor family signaling molecules. Nucleotide sequences of the RET gene and mRNA are known for a number of species, including human (e.g., as annotated under NCBI Gene ID: 5979).

Further non-limiting examples of applications of methods described herein include detection of hematological malignancy markers and panels thereof (e.g., including those to detect genomic rearrangements in lymphomas and leukemias), detection of sarcoma-related genomic rearrangements and panels thereof; and detection of IGH/TCR gene rearrangements and panels thereof for lymphoma testing.

In some embodiments, methods described herein relate to treating a subject having or diagnosed as having, e.g., cancer with a treatment for cancer. Subjects having cancer can be identified by a physician using current methods of diagnosing cancer. For example, symptoms and/or complications of lung cancer which characterize these conditions and aid in diagnosis are well known in the art and include but are not limited to, weak breathing, swollen lymph nodes above the collarbone, abnormal sounds in the lungs, dullness when the chest is tapped, and chest pain. Tests that may aid in a diagnosis of, e.g., lung cancer include, but are not limited to, x-rays, blood tests for high levels of certain substances (e.g., calcium), CT scans, and tumor biopsy. A family history of lung cancer, or exposure to risk factors for lung cancer (e.g., smoking or exposure to smoke and/or air pollution) can also aid in determining if a subject is likely to have lung cancer or in making a diagnosis of lung cancer.

Cancer can include, but is not limited to, carcinoma, including adenocarcinoma, lymphoma, blastoma, melanoma, sarcoma, leukemia, squamous cell cancer, small-cell lung cancer, non-small cell lung cancer, gastrointestinal cancer, Hodgkin's and non-Hodgkin's lymphoma, pancreatic cancer, glioblastoma, basal cell carcinoma, biliary tract cancer, bladder cancer, brain cancer including glioblastomas and medulloblastomas; breast cancer, cervical cancer, choriocarcinoma; colon cancer, colorectal cancer, endometrial carcinoma, endometrial cancer; esophageal cancer, gastric cancer; various types of head and neck cancers, intraepithelial neoplasms including Bowen's disease and Paget's disease; hematological neoplasms including acute lymphocytic and myelogenous leukemia; Kaposi's sarcoma, hairy cell leukemia; chronic myelogenous leukemia, AIDS-associated leukemias and adult T-cell leukemia lymphoma; kidney cancer such as renal cell carcinoma, T-cell acute lymphoblastic leukemia/lymphoma, lymphomas including Hodgkin's disease and lymphocytic lymphomas; liver cancer such as hepatic carcinoma and hepatoma, Merkel cell carcinoma, melanoma, multiple myeloma; neuroblastomas; oral cancer including squamous cell carcinoma; ovarian cancer including those arising from epithelial cells, sarcomas including leiomyosarcoma, rhabdomyosarcoma, liposarcoma, fibROS 1 arcoma, and osteosarcoma; pancreatic cancer; skin cancer including melanoma, stromal cells, germ cells and mesenchymal cells; pROS1tate cancer, rectal cancer; vulval cancer, renal cancer including adenocarcinoma; testicular cancer including germinal tumors such as seminoma, non-seminoma (teratomas, choriocarcinomas), stromal tumors, and germ cell tumors; thyroid cancer including thyroid adenocarcinoma and medullar carcinoma; esophageal cancer, salivary gland carcinoma, and Wilms' tumors. In some embodiments, the cancer can be lung cancer.

### Multiplex Methods

Methods described herein can be employed in a multiplex format. In embodiments of methods described herein, multiplex applications can include determining the nucleotide sequence contiguous to one or more known target nucleotide sequences. As used herein, “multiplex amplification” refers to a process that involves simultaneous amplification of more than one target nucleic acid in one or more reaction vessels. In some embodiments, methods involve subsequent determination of the sequence of the multiplex amplification products using one or more sets of primers. Multiplex can refer to the detection of between about 2-1,000 different target sequences in a single reaction. As used herein, multiplex refers to the detection of any range between 2-1,000, e.g., between 5-500, 25-1,000, or 10-100 different target sequences in a single reaction, etc. The term “multiplex” as applied to PCR implies that there are primers specific for at least two different target sequences in the same PCR reaction.

In some embodiments, target nucleic acids in a sample, or separate portions of a sample, can be amplified with a plurality of primers (e.g., a plurality of first and second target-specific primers). In some embodiments, the plurality of primers (e.g., a plurality of first and second target-specific primers) can be present in a single reaction mixture, e.g., multiple amplification products can be produced in the same reaction mixture. In some embodiments, the plurality of primers (e.g., a plurality of sets of first and second target-specific primers) can specifically anneal to known target sequences comprised by separate genes. In some embodiments, at least two sets of primers (e.g., at least two sets of first and second target-specific primers) can specifically anneal to different portions of a known target sequence. In some embodiments, at least two sets of primers (e.g., at least two sets of first and second target-specific primers) can specifically anneal to different portions of a known target sequence comprised by a single gene. In some embodiments, at least two sets of primers (e.g., at least two sets of first and second target-specific primers) can specifically anneal to different exons of a gene comprising a known target sequence. In some embodiments, the plurality of primers (e.g., first target-specific primers) can comprise identical 5′ tag sequence portions.

In embodiments of methods described herein, multiplex applications can include determining the nucleotide sequence contiguous to one or more known target nucleotide sequences in multiple samples in one sequencing reaction or sequencing run. In some embodiments, multiple samples can be of different origins, e.g., from different tissues and/or different subjects. In such embodiments, primers (e.g., tailed random primers) can further comprise a barcode portion. In some embodiments, a primer (e.g., a tailed random primer) with a unique barcode portion can be added to each sample and ligated to the nucleic acids therein; the samples can subsequently be pooled. In such embodiments, each resulting sequencing read of an amplification product will comprise a barcode that identifies the sample containing the template nucleic acid from which the amplification product is derived.

### Molecular Barcodes

In some embodiments, primers may contain additional sequences such as an identifier sequence (e.g., a barcode, an index), sequencing primer hybridization sequences (e.g., Rd1), and adapter sequences. In some embodiments the adapter sequences are sequences used with a next generation sequencing system. In some embodiments, the adapter sequences are P5 and P7 sequences for Illumina-based sequencing technology. In some embodiments, the adapter sequence are P1 and A compatible with Ion Torrent sequencing technology.

In some embodiments, as used herein, “molecular barcode,” “molecular barcode tag,” and “index” may be used interchangeably, and generally refer to a nucleotide sequence of a nucleic acid that is useful as an identifier, such as, for example, a source identifier, location identifier, date or time identifier (e.g., date or time of sampling or processing), or other identifier of the nucleic acid. In some embodiments, such molecular barcode or index sequences are useful for identifying different aspects of a nucleic acid that is present in a population of nucleic acids. In some embodiments, molecular barcode or index sequences may provide a source or location identifier for a target nucleic acid. For example, a molecular barcode or index sequence may serve to identify a patient from whom a nucleic acid is obtained. In some embodiments, molecular barcode or index sequences enable sequencing of multiple different samples on a single reaction (e.g., performed in a single flow cell). In some embodiments, an index sequence can be used to orientate a sequence imager for purposes of detecting individual sequencing reactions. In some embodiments, a molecular barcode or index sequence may be 2 to 25 nucleotides in length, 2 to 15 nucleotides in length, 2 to 10 nucleotides in length, 2 to 6 nucleotides in length. In some embodiments, a barcode or index comprise at least 2, 3, 4, 5, 6, 7, 8, 9, 10, 11, 12, 13, 14, 15, 16, 17, 18, 19, 20, 21, 22, 23, 24, or at least 25 nucleotides.

In some embodiments, when a population of tailed random primers is used in accordance with methods described herein, multiple distinguishable amplification products can be present after amplification. In some embodiments, because tailed random primers hybridize at various positions throughout nucleic acid molecules of a sample, a set of target-specific primers can hybridize (and amplify) the extension products created by more than 1 hybridization event, e.g., one tailed random primer may hybridize at a first distance (e.g., 100 nucleotides) from a target-specific primer hybridization site, and another tailed random primer can hybridize at a second distance (e.g., 200 nucleotides) from a target-specific primer hybridization site, thereby resulting in two amplification products (e.g., a first amplification product comprising about 100 bp and a second amplification product comprising about 200 bp). In some embodiments, these multiple amplification products can each be sequenced using next generation sequencing technology. In some embodiments, sequencing of these multiple amplification products is advantageous because it provides multiple overlapping sequence reads that can be compared with one another to detect sequence errors introduced during amplification or sequencing processes. In some embodiments, individual amplification products can be aligned and where they differ in the sequence present at a particular base, an artifact or error of PCR and/or sequencing may be present.

### Computer and Control Equipment

The systems provided herein include several components, including sensors, environmental control systems (e.g., heaters, fans), robotics (e.g., an XY positioner), etc. which may operate together at the direction of a computer, processor, microcontroller or other controller. The components may include, for example, an XY positioner, a liquid handling devices, microfluidic pumps, linear actuators, valve drivers, a door operation system, an optics assembly, barcode scanners, imaging or detection system, touchscreen interface, etc.

In some cases, operations such as controlling operations of a systems and/or components provided therein or interfacing therewith may be implemented using hardware, software or a combination thereof. When implemented in software, the software code can be executed on any suitable processor or collection of processors, whether provided in a single component or distributed among multiple components. Such processors may be implemented as integrated circuits, with one or more processors in an integrated circuit component. A processor may be implemented using circuitry in any suitable format.

A computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable, mobile or fixed electronic device, including the system itself.

In some cases, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. In other examples, a computer may receive input information through speech recognition or in other audible format, through visible gestures, through haptic input (e.g., including vibrations, tactile and/or other forces), or any combination thereof.

One or more computers may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools, and may be compiled as executable machine language code or intermediate code that is executed on a framework or virtual machine.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

In some embodiments, information related to the operation of the system (e.g., temperature, imaging or optical information, fluorescent signals, component positions (e.g., heated lid position, rotary valve position), liquid handling status, barcode status, bay access door position or any combination thereof) can be obtained from one or more sensors or readers associated with the system (e.g., located within the system), and can be stored in computer-readable media to provide information about conditions during a process (e.g., an automated library preparation process). In some embodiments, the readable media comprises a database. In some embodiments, said database contains data from a single system (e.g., from one or more bays). In some embodiments, said database contains data from a plurality of systems. In some embodiments, data is stored in a manner that makes it tamper-proof. In some embodiments, all data generated by the system is stored. In some embodiments, a subset of data is stored.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “ square,” as defined mathematically, to an extent typically achievable and achieved for the recited fabrication technique as would be understood by those skilled in the art or as specifically described. As another example, two or more fabricated articles that would described herein as being “ aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

What is claimed:

1. A cartridge, comprising:
a vessel comprising an inlet and a tapered cross-sectional shape, wherein the vessel has an internal working volume of at least 5 μl and less than or equal to 70 μl;
a microfluidic channel in fluid communication with the vessel for delivering a fluid to the vessel; and
an orifice positioned between the microfluidic channel and the vessel proximate the inlet to the vessel, wherein the orifice has a cross-sectional dimension of at least 10 microns and less than or equal to 500 microns.

2. A cartridge, comprising:
a vessel comprising an inlet;
an orifice positioned proximate the inlet to the vessel, wherein the orifice has a cross-sectional dimension of at least 10 microns and less than or equal to 500 microns;
a vent channel in fluid communication with the vessel and configured to receive a gaseous fluid from the vessel; and
a gas-permeable membrane configured to allow air to pass through the membrane while substantially preventing a liquid or vapor from passing across the membrane, wherein the vent channel is positioned between the gas-permeable membrane and the vessel.

3. A cartridge, comprising:
a vessel having an internal working volume of at least 5 μl and less than or equal to 70 μl, wherein the vessel has a longest dimension positioned along a first plane;
a microfluidic channel in fluid communication with the vessel and configured to deliver a fluid to the vessel, wherein the microfluidic channel has a longest dimension along a second plane, and
wherein the first plane is substantially perpendicular to the second plane.

4. A cartridge as in any one of the preceding claims, wherein the vessel is conical in shape.

5. A cartridge as in any one of the preceding claims, wherein the orifice is integral to the vessel.

6. A cartridge as in any one of the preceding claims, wherein the vessel has a taper angle of at least 20° and less than or equal to 45° with respect to vertical.

7. A cartridge as in any one of the preceding claims, wherein the vessel has an internal working volume of at least at least 5 μl and less than or equal to 70 μl.

8. A cartridge as in any one of the preceding claims, wherein the vessel is without a ledge.

9. A cartridge as in any one of the preceding claims, wherein the vessel has a tapered cross-sectional shape.

10. A cartridge as in any one of the preceding claims, wherein the cartridge comprises a first set of vessels and/or second set of vessels.

11. A cartridge as in any one of the preceding claims, wherein the first set of vessels and/or second set of vessels includes at least 2, 4, 6, 8, or 10 vessels and/or less than or equal to 20, 15, 10, or 5 vessels.

12. A cartridge as in any one of the preceding claims, wherein the vessel contains a reagent.

13. A cartridge as in any one of the preceding claims, wherein the reagent is substantially dry.

14. A cartridge as in any one of the preceding claims, wherein the reagent comprises a lyosphere.

15. A cartridge as in any one of the preceding claims, wherein the vessel contains more than one lyospheres.

16. A cartridge as in any one of the preceding claims, wherein the vessel comprises sidewalls.

17. A cartridge as in any one of the preceding claims, wherein the vessel is formed in a polymeric material.

18. A cartridge as in any one of the preceding claims, wherein the polymeric material comprises an injection-moldable polymer.

19. A cartridge as in any one of the preceding claims, wherein the cartridge comprises an orifice positioned between the microfluidic channel and the vessel proximate the inlet to the vessel, wherein the orifice has a cross-sectional dimension of at least 250 microns and less than or equal to 500 microns.

20. A cartridge as in any one of the preceding claims, wherein the orifice has a largest cross-sectional dimension that is smaller than a largest cross-sectional dimension of the microfluidic channel.

21. A cartridge as in any one of the preceding claims, wherein the orifice has a largest cross-sectional dimension that is smaller than a largest cross-sectional dimension of the inlet of the vessel.

22. A cartridge as in any one of the preceding claims, wherein the orifice is formed in a layer positioned between a layer comprising the microfluidic channel and a layer forming the vessel.

23. A cartridge as in any one of the preceding claims, wherein the gas-permeable membrane comprises a hydrophobic material.

24. A cartridge as in any one of the preceding claims, wherein the gas-permeable membrane comprises PTFE.

25. A cartridge as in any one of the preceding claims, wherein the gas-permeable membrane comprises polyethylene.

26. A cartridge as in any one of the preceding claims, wherein the gas-permeable membrane is positioned proximate the orifice.

27. A cartridge as in any one of the preceding claims, wherein the gas-permeable membrane is positioned at an outlet of the vessel.

28. A cartridge as in any one of the preceding claims, wherein the gas-permeable membrane is a part of a lid of the cartridge.

29. A cartridge as in any one of the preceding claims, wherein the gas-permeable membrane has an average pore size of at least 0.1 μm and less than or equal to 1 μm.

30. A cartridge as in any one of the preceding claims, wherein the gas-permeable membrane has a thickness of at least at least 1 μm and less than or equal to 1 mm.

31. A cartridge as in any one of the preceding claims, wherein the cartridge further comprises a removable protective strip positioned adjacent the gas-permeable membrane, wherein the removable protective strip provides a fluid barrier.

32. A cartridge as in any one of the preceding claims, wherein the removable protective strip is a foil.

33. A cartridge as in any one of the preceding claims, wherein the microfluidic channel has a cross-sectional dimension of at least 250 microns and less than or equal to 1 mm.

34. A cartridge as in any one of the preceding claims, wherein the cartridge comprises a first set of vessels and a first set of vessel channels, wherein the first set of vessel channels are microfluidic channels, and wherein each vessel channel is in fluid communication with a vessel.

35. A cartridge as in any one of the preceding claims, wherein the first set of vessel channels includes at least 2, 4, 6, 8, or 10 channels and/or less than or equal to 20, 15, 10, or 5 channels.

36. A cartridge as in any one of the preceding claims, wherein the cartridge comprises a second set of vessels and a second set of vessel channels, wherein the second set of vessel channels are microfluidic channels, and wherein each vessel channel is in fluid communication with a vessel.

37. A cartridge as in any one of the preceding claims, wherein the microfluidic channel(s) has/have a largest cross-sectional dimension of at least 20 microns and/or less than or equal to 1 mm.

38. A cartridge as in any one of the preceding claims, wherein the cartridge further interfaces with a lid that can be temperature-controlled.

39. A cartridge as in any one of the preceding claims, wherein the lid covers the vessels.

40. A cartridge as in any one of the preceding claims, wherein the lid forms a top portion of the vessel.

41. A cartridge as in any one of the preceding claims, wherein the heated lid is translucent or transparent.

42. A cartridge as in any one of the preceding claims, wherein the heated lid is configured to allow optical measurements to be taken therethrough.

43. A cartridge as in any one of the preceding claims, wherein the cassette is in communication with a temperature control device.

44. A cartridge as in any one of the preceding claims, wherein the temperature control device is configured to apply a first temperature to the lid.

45. A cartridge as in any one of the preceding claims, wherein the temperature control device comprises one or more thermal pads, thermoelectric components, and/or thermistors.

46. A cartridge as in any one of the preceding claims, wherein the vent channel is formed in a gap between a lid and a sidewall of the vessel.

47. A cartridge as in any one of the preceding claims, wherein the vent channel has a largest cross-sectional dimension of at least 50 μm and/or less than or equal to 750 μm.

48. A cartridge as in any one of the preceding claims, wherein the vent channel has a height and/or a width of at least 50 μm and/or less than or equal to 750 μm.

49. A cartridge as in any one of the preceding claims, wherein the vent channel has a length of at least 50 μm and/or less than or equal to 2 cm.

50. A cartridge as in any one of the preceding claims, wherein the cartridge further comprises an outlet channel positioned between the outlet and the vent channel.

51. A cartridge as in any one of the preceding claims, wherein the outlet channel is formed in a cover covering the cartridge.

52. A cartridge as in any one of the preceding claims, wherein the outlet channel has a largest cross-sectional dimension of at least 1 μm and/or less than or equal to 1 mm.

53. A cartridge as in any one of the preceding claims, wherein the outlet channel has a length of at least 1 mm and/or less than or equal to 1 cm.

54. A cartridge as in any one of the preceding claims, wherein the vessel is constructed and arranged to be heated by a thermal jacket that surrounds at least portion of the vessel.

55. A method for performing a reaction, comprising:
flowing a first fluid comprising a first reagent in a microfluidic channel;
introducing at least a portion of the first fluid to a vessel having an internal working volume and containing a second reagent to fill a portion, but not all, of the internal working volume of the vessel with the first fluid; and
reacting the first reagent with the second reagent, wherein during the reaction a ratio by volume of liquid to a gaseous fluid in the reaction vessel is at least 1 to 5 and less than or equal to 5 to 1.

56. A method as in any one of the preceding claims, comprising introducing the first fluid into the inlet of the vessel, wherein the inlet is positioned at a bottom portion of the vessel.

57. A method as in any one of the preceding claims, comprising introducing a volume of at least 5 microliters and less than or equal to 70 microliters of the first fluid into the vessel.

58. A method as in any one of the preceding claims, comprising allowing a gaseous fluid to pass through a vent channel in fluid communication with the vessel.

59. A method as in any one of the preceding claims, comprising allowing air to pass through a gas-permeable membrane in fluid communication with the vessel while substantially preventing a liquid or vapor from passing across the membrane.

60. A method as in any one of the preceding claims, comprising introducing a second fluid into the vessel, wherein the second fluid is a hydrophobic fluid.

61. A method as in any one of the preceding claims, wherein the second fluid has a volume of at least 1 microliter and less than or equal to 50 microliters.

62. A method as in any one of the preceding claims, comprising heating a lid of the vessel.

63. A method as in any one of the preceding claims, comprising condensing at least a portion of fluid in the vessel.

64. A method as in any one of the preceding claims, performing a chemical and/or biological reaction in the vessel at a pressure of at least 1 psi and less than or equal to 4 psi inside the vessel.

65. A method as in any one of the preceding claims, comprising heating the vessel.
